(19) United States
(12) Patent Application Publication
(10) Pub. No.: US 2023/0390474 A1
(43) Pub. Date: Dec. 7, 2023

(54) EXTRACORPOREAL DEVICE AND MATRIX FOR REMOVING AMMONIA FROM BIOLOGICAL FLUIDS, METHODS AND USES THEREOF

(71) Applicant: PLAS-FREE LTD, Nazareth (IL)

(72) Inventors: Zeev DVASHI, Kiryat Ono (IL); Nuha HIJAZI, D.N. Shimshon (IL)

(73) Assignee: PLAS-FREE LTD, Nazareth (IL)

(21) Appl. No.: 18/266,467

(22) PCT Filed: Dec. 9, 2021
(86) PCT No.: PCT/IL2021/051473

§ 371 Date: Jun. 9, 2023

Related U.S. Application Data
(60) Provisional application No. 63/123,851, filed on Dec. 10, 2020.

(51) Int. Cl. A61M 1/34; B01J 20/24; B01J 20/28; B01J 20/289; B01J 20/32; B01D 15/22; B01D 15/38

(52) U.S. Cl. CPC A61M 1/3486; A61M 1/3496; A61M 1/3401; B01J 20/24; B01J 20/28016; B01J 20/289; B01J 20/3295; B01J 20/3212; B01J 20/3257; B01J 20/3219; B01D 15/22; B01D 15/3804

(57) ABSTRACT

The present invention pertains to present invention relates to a device comprising conjugate/s, and uses thereof in depleting at least one amine, specifically ammonia from body fluids. The present disclosure further provides systems, apparatus, conjugates, plurality of conjugates, and methods. More specifically, the conjugate comprising a particle bonded to at least one linker comprising a chain of n carbon atoms covalently bonded to m carbonyl groups, and at least one trapping agent A covalently bonded to the m^{th} carbonyl group, (Formula I) wherein, n is an integer within the range of 5 to 15, and m is an integer within the range of 5 to 10, wherein trapping agent A is characterized by having the ability to capture or bind amine. In some optional embodiments, the amine is at least one of methylamine, dimethylamine or trimethylamine. In some embodiments, the linker of the conjugate of the disclosed device comprises a straight chain alkane and m carbonyl groups

### FIELD OF THE INVENTION

The present invention pertains to the field of plasmapheresis. More specifically, the present invention provides specific device and matrix for depleting ammonia from biological fluids, the resulting biological fluid that are devoid of ammonia, methods and uses thereof.

### BACKGROUND ART

References considered to be relevant as background to the presently disclosed subject matter are listed below:
[1] Treatment of hepatic encephalopathy by on-line hemodiafiltration: a case series study, Shinju Arata, Katsuaki Tanaka, Kazuhisa Takayama, Yoshihiro Moriwaki, Noriyuki Suzuki, Mitsugi Sugiyama & Kazuo Aoyagi, May 21, 2010;
[2] Extracorporeal Detoxification Using the Molecular Adsorbent Recirculating System for Critically Ill Patients with Liver Failure Steffen R. Mitzner, Jan Stange, Sebastian Klammt, Piotr Peszynski, Reinhardt Schmidt and Gabriele Noldge-Schomburg Jasn February 2001, 12 (suppl 1) S75-S82;
[3] Ion-exchange resins in the treatment of anuria b. m. evans. et al Lancet 1953;
[4] Extracorporeal methods of reducing high blood ammonia levels H. D. Ritchie, D. M. Davies, J. M. Godfrey, P. Fan, R. G. S. Johns, and J. Perrin, Gut, 1962.
[5] Hyperkalemia in chronic kidney disease, Renato Watanabe-Rev. Assoc. Med. Bras. vol. 66 supl. 1 São Paulo 2020 Epub Jan. 13, 2020.
[6] Effects of potassium adsorption filters on the removal of ammonia from blood products Hiroshi Fujita, kYoko Shiotani, et al, March 2018;
[7] Blood Ammonia Reduction by Potassium Exchange Resin Experimentation in Eck-Fistula Dogs, GEORGE D. ZUIDEMA et al 0.1963.
[8] Membrane unit and device for cleansing blood, U.S. Pat. No. 4,183,811A;
[9] Liver support system, WO2014079681A2; System and method for extracorporeal blood treatment, WO2016205221A1
[10] WO2004014315A2;
[11] U.S. Pat. No. 3,963,613A;
[12] JP2008093244A;
[13] CN100486651C;
[14] CN109692372A.

Acknowledgement of the above references herein is not to be inferred as meaning that these are in any way relevant to the patentability of the presently disclosed subject matter.

### BACKGROUND

Hepatic encephalopathy is a potentially reversible, or progressive, neuropsychiatric syndrome characterized by changes in cognitive function, behavior, and personality, as well as by transient neurological symptoms and characteristic electroencephalographic patterns associated with acute and chronic liver failure. Hepatic encephalopathy is a frequent complication of cirrhosis that is usually observed in association with severe hepatic insufficiency. The characteristic presentation is the development of acute encephalopathy with an abrupt decline in the level of consciousness, manifested as confusion or coma. Frequently, a precipitating factor can be identified. The treatment of the episode is directed toward the correction of the precipitating factor. Once the precipitating condition is resolved the encephalopathy also typically disappears, with the patient recovering to his or her previous state. However, in patients with low reserves of hepatic function, the hepatic encephalopathy can be a chronic condition. The low reserve predisposes the patient to development of spontaneous hepatic encephalopathy. One of the main particles reopenable to the pathophysiology of HE is ammonia.

Ammonia, a byproduct of the metabolism of nitrogen-containing compounds, is neurotoxic at elevated concentrations. The liver clears almost all of the portal vein ammonia, converting it into glutamine and urea preventing entry into the systemic circulation. However, glutamine is metabolized in mitochondria yielding glutamate and ammonia, and glutamine-derived ammonia may interfere with mitochondrial function leading to astrocytes dysfunction. The increase in blood ammonia in advanced liver disease is a consequence of impaired liver function and of shunting of blood around the liver. Muscle wasting, a common occurrence in these patients, also may contribute since muscle is an important site for extrahepatic ammonia removal. In addition to direct neurotoxicity, low-grade astrocyte swelling may contribute to brain dysfunction. The enzyme glutamine synthetase (present in the endoplasmic reticulum of astrocytes) is responsible for the conversion of the ammonia to glutamine. As glutamine acts as osmolyte, water moves inside the astrocyte causing low-grade cerebral edema and a predominantly neuroinhibitory state (that is, slowing of mental processes) is pathognomonic of HE, which is associated with chronic liver disease.

Treatment of encephalopathy by artificial liver support (ALS) and a combination of hemodiafiltration (HDF) has been previously demonstrated [1].

Steffen R. et al., [2] demonstrates molecular adsorbent recirculating system (MARS) represents a cell-free, extracorporeal, liver assistance method for the selective removal of albumin-bound substances. Moreover, it enables the removal of excess water and water-soluble substances via an inbuilt dialysis step.

Still further, Evans. et al., [3] demonstrates treatment of Anuria using a method involving introduction of carboxylic ion-exchange resin charged with ammonium ion, both orally and by retention enemata. A satisfactory exchange for potassium ions, with a definite fall of the serum-potassium level has been reported.

The use of Ion-exchange resins, in particular a British resin ZK.225 having a 20% divinyl-benzene linkage, was reported to be effective [4]. When passed from an artery directly through an autoclaved resin column to a vein, significant amounts of ammonia were removed from the blood of a dog with pronounced hyper ammonia.

It has been recently demonstrated that Hyperkalemia increases the risk of cardiac arrhythmia episodes and sudden death [5]. The control of potassium elevation is thus essential for reducing the mortality rate in this population.

Fujita et al., demonstrate the effect of potassium adsorption filters on the removal of ammonia from blood products [6]. This publication demonstrates potassium adsorption filter (PAF) that is available for bedside use for removal of potassium ions from packed red blood cell (RBC) solutions. The disclosed method is reported as applicable for patients requiring rapid, massive transfusion and can reduce the levels of ammonia.

The used of a potassium-cycle exchange resin to lower blood ammonia in Eck-fistula dogs has been previously reported [7]. The disclosed resin has the advantage of exchanging potassium ions for ammonium and sodium ions.

U.S. Pat. No. 4,183,811A [8] discloses a membrane unit and apparatus for removing toxic metabolites and metabolites normally present in urine from blood.

WO2014079681A2 [9] discloses an artificial, extracorporeal system for liver replacement and/or assistance, comprising a liver dialysis device for conducting hemodialysis on a patient suffering from liver failure. The system disclosed therein is characterized as comprising a first standard hollow fiber membrane dialyzer which does not allow passage of an essential amount of albumin over the membrane wall and which is perfused with the patient's blood, and a second hollow fiber membrane dialyzer which allows the passage of essential but defined amounts of albumin over the membrane wall and which receives the blood of the first standard hemodialyzer. The filtrate space is closed off from the lumen space of the hollow fibers and is populated by adsorbent material which may comprise one or more different adsorbents.

WO2016205221A1 [10] discloses extracorporeal filtration and detoxification system and method for separating ultrafiltrate from cellular components of blood. The methods involve treating the ultrafiltrate independently of the cellular components in a recirculation circuit, recombining treated ultrafiltrate and the cellular components, and returning whole blood to the patient.

WO2004014315A2 [11] demonstrates method for removing from a patient's blood and/or specific plasma fraction containing substances within a specific molecular weight range.

U.S. Pat. No. 3,963,613A [12], discloses a blood purification means, whereby blood of a patient to be treated is led around an external circuit connecting to the patient's blood stream and is brought into direct or indirect contact with a fumarate solution. The blood is further contacted with an enzymic preparation, which is suitably an aspartase preparation, which catalyzes a reaction of L-aspartic acid formation from fumaric acid and ammonia. The purification means may further include a preliminary stage or stages whereat an unrequired substance in a patients blood is decomposed to a non-toxic substance and ammonia subsequently convertable to aspartic acid and may also include a low molecular sieve means preventing re-entry of aspartic acid produced into the patient's blood stream.

JP2008093244A [13] discloses a method capable of efficiently removing ammonia contained in a liquid such as blood or plasma by using silica gel. It should be noted however, that the filters disclosed by this publication are not specific for ammonia but can also deplete other small particles such as lipopolysaccharide. Moreover, most of the currently available raisins are based on ion exchange materials that can remove ammonia from plasma but are not specific to ammonia only. It should be understood that since ammonia is cationic in physiologic pH, it can be bound by any cation exchangers that are not specific only to ammonia. There is therefore need for resins that are specifically designated to remove only ammonia from the plasma, as disclosed by the present invention.

CN100486651C [14] demonstrates a multiple organ function support system, formed by a body, an external blood circuit, a plasma separating-adsorbing circuit, an albumin circuit, a dialysate circuit, a feeding circuit and an operating system. This publication further discloses systems for elimination of inflammation medium, toxin and small molecule materials (e.g., blood ammonia) from body fluids of patients suffering from multiple organ function obstacle complex symptom (MODS). This publication discloses a lung membrane oxygen generator that replaces the air exchange function of lung and uses thereof in reversal breath exhaustion.

CN109692372A [15] discloses a five layers blood perfusion device and blood perfusion method. More specifically, an anticoagulant layer of gel micro-ball, β2-microglobulin adsorption layer, urea decomposition layer, Ammonia adsorption layer and activated carbon adsorption layer are successively arranged along direction of flow of blood in the perfusion device ontology. The blood perfusion method provided therein is directed at purifying of up to 7000 ug of ammonia from blood. However, removal of much higher quantities of ammonia is required.

There is therefore need in the art for effective means ad methods for depleting ammonia from body fluids.

### SUMMARY OF THE INVENTION

In a first aspect of the present invention relates to a device comprising:
a housing having at least one fluid inlet port, and at least one fluid outlet port;
the housing including at least one chamber, said at least one chamber defining a control volume in fluid communication with the at least one fluid inlet port and the at least one fluid outlet port. In some embodiments, the control volume accommodating at least one of a conjugate/s, a plurality of conjugates or at least one composition comprising the conjugates or plurality of conjugates. More specifically, the conjugate comprising a particle bonded to at least one linker comprising a chain of n carbon atoms covalently bonded to m carbonyl groups, and at least one trapping agent A covalently bonded to the m^{th} carbonyl group,

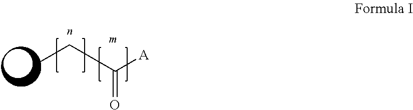

wherein, n is an integer within the range of 5 to 15, and m is an integer within the range of 5 to 10, wherein trapping agent A is characterized by having the ability to capture or bind amine. In some optional embodiments, the amine is at least one of methylamine, dimethylamine or trimethylamine.

In some embodiments, the linker of the conjugate of the disclosed device comprises a straight chain alkane and m carbonyl groups.

In some further embodiments, the straight chain alkane is saturated or unsaturated.

Still further, in some embodiments, the straight chain alkane of the conjugate of the disclosed device is unsaturated.

In certain embodiments, the straight chain contains between 1 to 3 double bonds.

Still further, in some embodiments of the disclosed device, t the amine is ammonia.

In yet some further embodiments, the linker of the conjugate of the disclosed device is covalently linked to the trapping agent in a way that the m^{th} carbonyl is linked via straight linkage

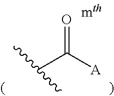

or through another short alkane chain

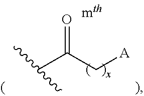

wherein X is an integer within the range of 1 to 3.

In some embodiments, the trapping agent of the conjugate of the disclosed device, is a strong acid, capable of capturing ammonia.

Still further, in some embodiments, the strong acid is sulfuric acid or any derivates thereof.

In yet some further embodiments, the length of the straight chain alkanes (n) is 15.

In certain embodiments, the conjugate/s of the disclosed device comprise/s a particle bonded to at least one linker comprising a chain of 15 carbon atoms covalently bonded to between 5 to 10 carbonyl groups (m), and an acid A covalently bonded to the m^{th} carbonyl group, the conjugate having the structural formula II:

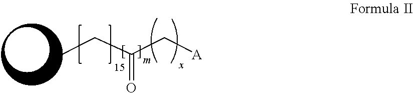

wherein x is between 0 to 3.

Still further, in some embodiments, the conjugate/s of the disclosed device comprise/s a particle bonded to at least one linker comprising a chain of 15 carbon atoms covalently bonded to between 5 to 10 carbonyl groups (m), and a sulfonic acid covalently bonded to the m^{th} carbonyl group, the conjugate having the structural formula III:

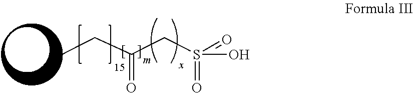

wherein x is between 0 to 3.

In some embodiments, the particle and the linker are covalently connected, and wherein the linkage is a covalent linkage via amino group as presented in Formula IV:

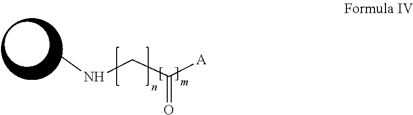

In certain embodiments, the of the conjugate of the disclosed device, is having the structural formula V. More specifically, the conjugate comprising a particle covalently bonded to at least one linker comprising a chain of 15 carbon atoms covalently bonded to m carbonyl groups, and a sulfonic acid covalently bonded to the m^{th} carbonyl group

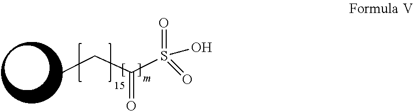

wherein m is an integer between 5 to 10.

In some embodiments, the particle is a resin bead. In some embodiments, the particle may be an agarose bead, and therefore, the resin bead is in some embodiments the agarose resin may comprise between about 2% to 10% agarose. Still further, in some embodiments the resin bead may comprise between 3% to 9% agarose, still further in some embodiments, the resin bead may comprise between 4% to 8% agarose. According to further specific any non-limiting embodiments, the resin bead optionally comprises at least 4% of agarose.

In yet some further embodiments, the resin bead size ranges between 40 to 170 μm. In yet some further embodiments, the device comprises a first barrier member and a second barrier member, longitudinally spaced from one another via said control volume, the first barrier member and the second barrier member each being configured for permitting fluid flow in one direction through the respective barrier member, and for blocking fluid flow in an opposite direction through the respective barrier member. In some embodiments, the first barrier member and the second barrier member are installed in the device in a manner to permit fluid flow through the device from the at least one fluid inlet port to the at least one fluid outlet port, and for concurrently blocking fluid flow from the fluid outlet port to the fluid inlet port.

In yet some further embodiments of the disclosed device, each one of the first barrier member and the second barrier member comprises a membrane made from suitable material.

Still further, the housing comprises an outer casing, an inlet end cap and an outlet end cap, wherein the outer casing comprises an outer wall extending longitudinally between an inlet end and an outlet end of the outer casing. The inlet end cap is configured for being sealingly mounted to the inlet end, and the outlet end cap is configured for being sealingly mounted to the outlet end.

In yet some further embodiments of the device according to the present disclosure, the inlet end cap, the outlet end cap, and the outer casing are each made from suitable medically compatible materials.

In certain embodiments, the inlet end cap is configured as a self-locking cap with respect to the outer casing and configured for enabling the inlet end cap to be sealingly locked in place with respect to the outer casing.

Still further, in some embodiments, the disclosed device comprising a first self-locking arrangement configured for enabling self-locking of the inlet end cap with respect to the outer casing.

In some embodiments, the first self-locking arrangement comprises a plurality of first wedge elements and a first flange arrangement. Still further, the first wedge elements are provided in the inlet end cap, and wherein the first flange arrangement is provided in the outer casing longitudinally spaced from the inlet end by a first spacing, and wherein the first wedge elements are configured for cooperating with a first flange stop arrangement to provide self-locking of the inlet end cap with respect to the housing.

In yet some further embodiments of the disclosed device, each of the first wedge element is projecting in a longitudinal direction away from a free end of the first end cap.

In some embodiments, the first spacing is sufficient such as to ensure that when the inlet end cap is fully engaged with the outer casing, the respective free end of the inlet end cap is in abutting contact with the first flange arrangement.

In some embodiment of the disclosed device, the first flange stop arrangement comprises a plurality of first stop elements corresponding to the plurality of first wedge elements. Still further, each of the first stop element operates to prevent disengagement of the inlet end cap from the outer casing when the respective first wedge element is in abutting contact therewith.

In some embodiments of the disclosed device, the first flange stop arrangement comprises a first flange including a plurality of first cutouts corresponding to the first wedge elements. Still further, each said first cutout has a circumferential length and an axial depth sufficient to enable accommodating a respective the first wedge element therein in locked configuration.

In some embodiments, the outlet end cap is configured as a self-locking cap with respect to the outer casing and configured for enabling the outlet end cap to be sealingly locked in place with respect to the outer casing.

In some embodiments, the device of the present disclosure comprises a second self-locking arrangement configured for enabling self-locking of the outlet end cap with respect to the outer casing.

In more specific embodiments of the disclosed device, the second self-locking arrangement comprises a plurality of second wedge elements and a second flange arrangement. The second wedge elements are provided in the outlet end cap, and wherein the second flange arrangement is provided in the outer casing longitudinally spaced from the outlet end by a second spacing. Still Further, the second wedge elements are configured for cooperating with a second flange stop arrangement to provide self-locking of the outlet end cap with respect to the housing.

In some embodiments, each of the second wedge element is projecting in a longitudinal direction away from a free end of the second end cap.

In some further embodiments of the disclosed device, the second spacing is sufficient such as to ensure that when the outlet end cap is fully engaged with the outer casing, the respective free end of the outlet end cap is in abutting contact with the second flange arrangement.

In some further embodiments, the second flange stop arrangement comprises a plurality of second stop elements corresponding to the plurality of second wedge elements. Still further, each of the second stop element operates to prevent disengagement of the outlet end cap from the outer casing when the respective second wedge element is in abutting contact therewith.

In some embodiments, the second flange stop arrangement comprises a second flange including a plurality of second cutouts corresponding to the second wedge elements. Still further, each said second cutout has a circumferential length and an axial depth sufficient to enable accommodating a respective the second wedge element therein in locked configuration.

In some embodiments of the disclosed device, the control volume is between about 250 ml and about 350 ml. In yet some further embodiments, the control volume is about 250, 255, 260, 265, 270, 275, 280, 285, 290, 295, 300, 305, 310, 315, 320, 325, 330, 335, 340, 345, 350 ml. Still further, in some embodiments the control volume is between about 257 ml and about 326 ml.

In some embodiments, the device of the present disclosure is configured for use in depleting at least one amine from at least one liquid substance.

In some specific embodiments, the amine depleted by the disclosed device is ammonia.

In yet some further embodiments, the device of the invention is configured for depleting at least one amine from a liquid substance, that may be a mammalian body fluid. Thus, in some embodiments, the device is for use in depleting ammonia from mammalian body fluid/s.

In some specific embodiments, the conjugate of the disclosed device is having the structural formula V, the conjugate comprising a particle covalently bonded to at least one linker comprising a chain of 15 carbon atoms covalently bonded to m carbonyl groups, and a sulfonic acid covalently bonded to the m^{th} carbonyl group

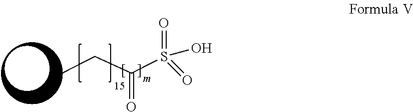

wherein m is an integer between 5 to 10.

A further aspect of the present disclosure relates to a system comprising:
at least one device as defined by the present disclosure;
an apheresis machine;
a blood mixing reservoir; and
a conduit system.

In some embodiments, the conduit system comprises a first conduit configured for providing selective fluid communication between the apheresis machine and a body of a subject in need thereof, to thereby enable blood to flow to the apheresis machine from the body of a subject in need thereof.

In some embodiments, the conduit system comprises a second conduit configured for providing fluid communication from a plasma outlet of the apheresis machine and the at least one device, to thereby enable plasma, separated from blood by the apheresis machine, to flow into the at least one device.

Still further, in some embodiments, the conduit system comprises a third conduit configured for providing fluid communication from the at least one device to the blood mixing reservoir, to thereby enable processed plasma, treated by the at least one device, to flow into the blood mixing reservoir.

In certain embodiments, the conduit system comprises a fourth conduit configured for providing fluid communication from a blood products outlet of the apheresis machine to the blood mixing reservoir, to thereby enable other blood products separated from blood by the apheresis machine, to flow into the blood mixing reservoir.

In yet some further embodiments, the conduit system comprises a fifth conduit configured for providing selective fluid communication between the blood mixing reservoir and the body of a subject in need thereof, to thereby enable treated blood to flow from the blood mixing reservoir to the body of a subject in need thereof.

It should be noted that in some embodiments of the disclosed systems, a subject in need thereof is a subject suffering from at least one disorder associated with elevated blood ammonia levels. Specifically, any of the disorders discussed in connection with other aspects of the invention.

In some embodiments the disclosed system comprises a plurality of the devices disclosed herein, interconnected in series with respect to one another.

In yet some further embodiments, the disclosed systems comprise a plurality of said devices, interconnected in parallel with respect to one another via an inlet manifold coupled to each respective fluid inlet port, and via an outlet manifold coupled to each respective fluid outlet port.

In some further embodiments, the disclosed system comprises a first plurality of groups of the devices, the groups being interconnected in parallel with respect to one another via an inlet manifold coupled to each respective fluid inlet port, and via an outlet manifold coupled to each respective fluid outlet port, and wherein each of the group comprising a respective second plurality of the devices interconnected in series with respect to one another within the respective group.

A further aspect of the present disclosure relates to a battery for use in depleting ammonia from mammalian body fluid/s, comprising a plurality of devices as defined by the present disclosure.

A further aspect provided by the present disclosure relates to a battery for use in depleting ammonia from mammalian body fluid/s, comprising a plurality of devices defined by the present disclosure.

A further aspect of the present disclosure relates to an extracorporeal apparatus comprising at least one conjugate, or at least one device comprising the conjugate, or connected to the at least one device or battery of devices. More specifically, the conjugate comprising a particle bonded to at least one linker comprising a chain of n carbon atoms covalently bonded to m carbonyl groups, and at least one trapping agent A covalently bonded to the m^{th} carbonyl group,

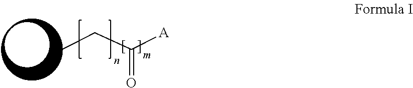

wherein, n is an integer within the range of 5 to 15, and m is an integer within the range of 5 to 10, wherein trapping agent A is characterized by having the ability to capture or bind amine Optionally, the amine is at least one of methylamine, dimethylamine or trimethylamine. Still further, the device comprising:
a housing having at least one fluid inlet port, and at least one fluid outlet port;
the housing including at least one chamber, the at least one chamber defining a control volume in fluid communication with the at least one fluid inlet port and the at least one fluid outlet port;
the control volume accommodating said at least one of a conjugate, a plurality of conjugates or at least one composition comprising the conjugates or plurality of conjugates.

A further aspect relates to an extracorporeal apparatus comprising at least one conjugate, or at least one device comprising the conjugate. In some embodiments, the extracorporeal apparatus may be connected to such at least one device or battery of devices. In more specific embodiments, the conjugate of the extracorporeal apparatus of the present disclosure may comprise a particle bonded to at least one linker comprising a chain of n carbon atoms covalently bonded to m carbonyl groups, and at least one trapping agent A covalently bonded to the m^{th} carbonyl group,

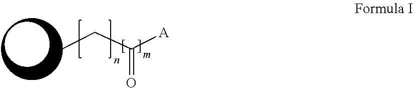

wherein, n is an integer within the range of 5 to 15, and m is an integer within the range of 5 to 10. More specifically, the trapping agent A is characterized by having the ability to capture or bind amine, optionally, the amine is at least one of methylamine, dimethylamine or trimethylamine. In yet some further embodiments, the device comprised within o connected to the extracorporeal apparatus may comprise:
a housing having at least one fluid inlet port, and at least one fluid outlet port;
the housing including at least one chamber, said at least one chamber defining a control volume in fluid communication with the at least one fluid inlet port and the at least one fluid outlet port. The control volume accommodating the at least one of a conjugates, a plurality of conjugates or at least one composition comprising the conjugates or plurality of conjugates.

In some embodiments, of the extracorporeal apparatus of the present disclosure o, the device is as defined by the present disclosure, and the battery is as define by the present disclosure. In some embodiments, the conjugate/s, the plurality of conjugates or composition, the device and the battery used for the extracorporeal apparatus is as define by the present disclosure.

In some embodiments, the extracorporeal apparatus of the present disclosure is applicable for use in depleting ammonia from mammalian body fluid/s.

A further aspect of the present disclosure relates to a conjugate having the structural formula I. More specifically, the conjugate comprising a particle bonded to at least one linker comprising a chain of n carbon atoms covalently bonded to m carbonyl groups, and at least one trapping agent A covalently bonded to the m^{th} carbonyl group;

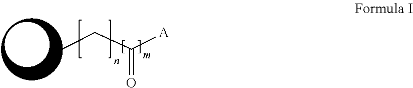

wherein, n is an integer within the range of 5 to 15, and m is an integer within the range of 5 to 10, wherein trapping agent A is characterized by having the ability to capture or bind amine Optionally, the amine is at least one of methylamine, dimethylamine or trimethylamine. Still further, in some embodiments, having the ability to capture is meant, binds and/or captures at least one amine.

In some embodiments, the linker of the disclosed conjugate comprises a straight chain alkane and m carbonyl groups.

Still further, in some embodiments, the straight chain alkane of the disclosed conjugate is saturated or unsaturated.

In some embodiments, the straight chain alkane is unsaturated.

In yet some further embodiments, the straight chain contains between 1 to 3 double bonds.

Still further, in some embodiments, trapping agent A of the conjugate the present disclosure is having the ability to capture and/or binds at least one amine, specifically, the amine is ammonia.

In yet some further embodiments of the disclosed conjugate, the linker is covalently linked to the trapping agent in a way that the m^{th} carbonyl is linked via straight linkage (

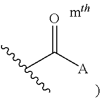

or through another short alkane chain

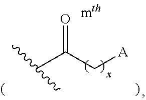

wherein X is an integer within the range of 1 to 3.

Still further, in some embodiments, the trapping agent of the disclosed conjugate is a strong acid, capable of capturing ammonia.

In some further embodiments, the strong acid is sulfuric acid or any derivates thereof.

In some embodiments, the length of the straight chain alkanes (n) is 15.

Still further, in some embodiments, the conjugate of the present disclosure comprises a particle bonded to at least one linker comprising a chain of 15 carbon atoms covalently bonded to between 5 to 10 carbonyl groups (m), and an acid A covalently bonded to the m^{th} carbonyl group, the conjugate having the structural formula II:

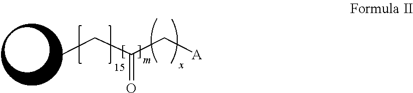

wherein x is between 0 to 3.

In yet some further embodiments, the comprising a particle bonded to at least one linker comprising a chain of 15 carbon atoms covalently bonded to between 5 to 10 carbonyl groups (m), and a sulfonic acid covalently bonded to the m^{th} carbonyl group, the conjugate having the structural formula III:

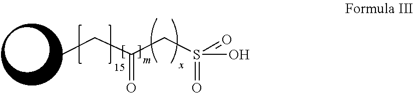

wherein x is between 0 to 3.

In some embodiments of the conjugate disclosed herein, the particle and the linker and covalently connected, and wherein the linkage is a covalent linkage via amino group as presented in Formula IV:

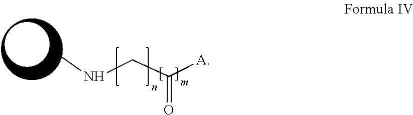

Still further, in some embodiments, the conjugate disclosed by the present disclosure is having the structural formula V, the conjugate comprising a particle covalently bonded to at least one linker comprising a chain of 15 carbon atoms covalently bonded to m carbonyl groups, and a sulfonic acid covalently bonded to the m^{th} carbonyl group

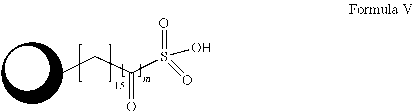

wherein m is an integer between 5 to 10.

In some embodiments, the particle is a resin bead. In some embodiments, the particle may be an agarose bead, and therefore, the resin bead is in some embodiments the agarose resin may comprise between about 2% to 10% agarose. Still further, in some embodiments the resin bead may comprise between 3% to 9% agarose, still further in some embodiments, the resin bead may comprise between 4% to 8% agarose. In some further embodiments, the particle of the disclosed conjugate is a resin bead. Still further, the resin bead optionally comprises at least 4% of agarose.

In some embodiments of the disclosed conjugate, the resin bead size ranges between 40 to 170 μm.

A further aspect of the present disclosure relates to a plurality of conjugates or any composition comprising said plurality of conjugates. Each conjugate comprises a particle, at least one linker and at least one trapping agent A, or any derivative or analog thereof, the conjugate comprising a particle bonded to at least one linker comprising a chain of n carbon atoms covalently bonded to m carbonyl groups, and at least one trapping agent A covalently bonded to the m^{th} carbonyl group;

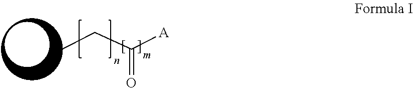

wherein, n is an integer within the range of 5 to 15, and m is an integer within the range of 5 to 10, wherein trapping agent A is characterized by having the ability to capture or bind amine Optionally, the amine is at least one of methylamine, dimethylamine or trimethylamine.

In some embodiments, the conjugate of the plurality of conjugate is any of the conjugates disclosed by the present disclosure.

In some embodiments, the plurality of conjugates as disclosed herein is for use in depleting at least one amine from at least one liquid substance.

In some embodiments of the disclosed plurality of conjugates, the amine is ammonia.

In some embodiments, the liquid substance is a mammalian body fluid.

Still further, in some other embodiments, the plurality of conjugates is for use in depleting ammonia from mammalian body fluid/s.

A further aspect of the resent disclosure relates to a method for depleting at least one amine from a liquid substance. More specifically, the method comprising the steps of: In a first step (i), subjecting the liquid substance to affinity-depletion procedure specific for the at least one amine. The next step (ii) involves recovering the at least one amine-depleted liquid obtained in step (i). In some embodiments, the affinity-depletion procedure comprises contacting the liquid substance with an effective amount of at least one conjugate, a plurality of conjugates or with a composition comprising the conjugate or plurality of conjugates, or applying the liquid substance on a device, battery, or extracorporeal apparatus comprising the conjugates of the present disclosure. In more specific embodiments, each conjugate comprises a particle bonded to at least one linker comprising a chain of n carbon atoms covalently bonded to m carbonyl groups, and at least one trapping agent A covalently bonded to the m^{th} carbonyl group,

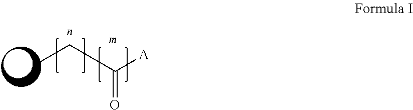

wherein, n is an integer within the range of 5 to 15, and m is an integer within the range of 5 to 10, wherein trapping agent A is characterized by having the ability to capture or bind the amine. Optionally, the amine is at least one of methylamine, dimethylamine or trimethylamine.

In some embodiments, the liquid substance used in the methods of the present disclosure is a mammalian body fluid/s or any products thereof.

In yet some further embodiments, the methods of the invention are used for depleting at least one amine from any liquid substance. In some embodiments, the amine is ammonia. Thus, in some embodiments, the methods of the present disclosure are for use in depleting ammonia from a mammalian body fluid.

In should be noted that in some embodiments, any of the conjugate/s, plurality of conjugates or composition, device and/or battery and/or apparatus used by the methods discussed herein ae as defined by the invention.

A further aspect of the invention relates to a method for depleting at least one amine from body fluid/s of a subject in need thereof. More specifically, the method may comprise contacting the body fluid with an effective amount of conjugates, a plurality of conjugates or a composition thereof, or within a device or battery comprising the conjugate, or alternatively, with an extracorporeal apparatus that comprises the conjugate o device described herein or connected to the conjugate or device disclosed herein. It should be noted that each conjugate comprises a particle bonded to at least one linker comprising a chain of n carbon atoms covalently bonded to m carbonyl groups, and at least one trapping agent A covalently bonded to the m^{th} carbonyl group,

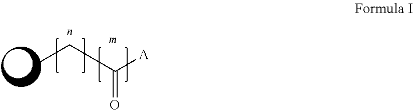

wherein, n is an integer within the range of 5 to 15, and m is an integer within the range of 5 to 10, wherein trapping agent A is characterized by having the ability to capture or bind amine Optionally, the amine is at least one of methylamine, dimethylamine or trimethylamine. The next step involves recovering the amine-free body fluid, and optionally, re introducing the body fluid to the subject in need.

In yet some further specific and non-limiting embodiments, the method may comprise the use of an extracorporeal procedure. More specifically, such method may comprise the steps of:

First in step (i), transferring body fluids of the subject into an extracorporeal apparatus. The next step (ii), involves subjecting the body fluid to affinity depletion procedure specific for at least one amine, wherein said depletion is performed before, during or after blood is being transferred into and out-off said apparatus, thereby obtaining an extracorporeal body fluid of said subject depleted in at least one amine.

The next step (iii) involves reintroducing or returning said body fluid obtained in step (ii) to the subject. As indicated above, the affinity-depletion procedure comprises contacting the body fluid of the subject with an effective amount of the conjugates, a plurality of conjugates or a composition thereof comprised within said extracorporeal apparatus, or within a device or battery connected to the extracorporeal apparatus. Each conjugate comprises a particle bonded to at least one linker comprising a chain of n carbon atoms covalently bonded to m carbonyl groups, and at least one trapping agent A covalently bonded to the m^{th} carbonyl group,

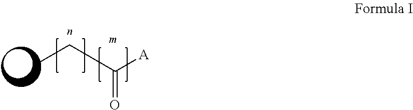

wherein, n is an integer within the range of 5 to 15, and m is an integer within the range of 5 to 10, wherein trapping agent A is characterized by having the ability to capture or bind amine Optionally, said amine is at least one of methylamine, dimethylamine or trimethylamine.

In some embodiment, the conjugate, plurality of conjugates or composition, device, battery and apparatus used by the methods of the invention are any of those disclosed by the present disclosure.

In some embodiments, the conjugate used by the methods of the present disclosure is having the structural formula V, the conjugate comprising a particle covalently bonded to at least one linker comprising a chain of 15 carbon atoms covalently bonded to m carbonyl groups, and a sulfonic acid covalently bonded to the m^{th} carbonyl group

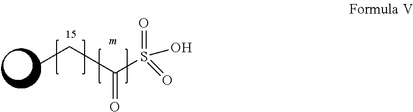

wherein m is an integer between 5 to 10.

A further aspect of the present disclosure relates to a method for the treatment, prevention, prophylaxis, amelioration, inhibition of disorders associated with elevated blood ammonia levels or pathologic condition associated therewith in a subject in need thereof by depleting ammonia from body fluid/s of a subject in need thereof.

More specifically, the therapeutic methods disclosed herein may comprise contacting the body fluid of the treated subject with an effective amount of conjugates, a plurality of conjugates or a composition thereof, or within a device or battery comprising the conjugate, or alternatively, with an extracorporeal apparatus that comprises the conjugate o device described herein or connected to the conjugate or device disclosed herein. It should be noted that each conjugate comprises a particle bonded to at least one linker comprising a chain of n carbon atoms covalently bonded to m carbonyl groups, and at least one trapping agent A covalently bonded to the m^{th} carbonyl group,

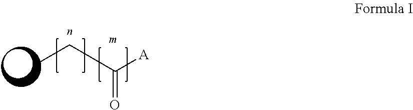

wherein, n is an integer within the range of 5 to 15, and m is an integer within the range of 5 to 10, wherein trapping agent A is characterized by having the ability to capture or bind amine Optionally, the amine is at least one of methylamine, dimethylamine or trimethylamine. The next step involves recovering the amine-free body fluid, and optionally, reintroducing the body fluid to the treated subject.

In yet some further specific and non-limiting embodiments, the methods may comprise the use of an extracorporeal procedure. More specifically, such method may comprise the steps of:

First in step (i), transferring body fluids of the subject into an extracorporeal apparatus. The next step (ii), involves subjecting the body fluid to affinity depletion procedure specific for at least one amine, wherein said depletion is performed before, during or after blood is being transferred into and out-off said apparatus, thereby obtaining an extracorporeal body fluid of the treated subject depleted in at least one amine.

The next step (iii) involves reintroducing or returning said body fluid obtained in step (ii) to the subject. As indicated above, the affinity-depletion procedure comprises contacting the body fluid of the subject with an effective amount of conjugates, a plurality of conjugates or a composition thereof comprised within said extracorporeal apparatus, or within a device or battery connected to the extracorporeal apparatus. Each conjugate comprises a particle bonded to at least one linker comprising a chain of n carbon atoms covalently bonded to m carbonyl groups, and at least one trapping agent A covalently bonded to the m^{th} carbonyl group,

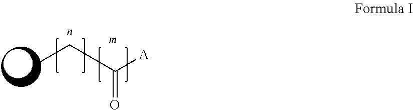

wherein, n is an integer within the range of 5 to 15, and m is an integer within the range of 5 to 10, wherein trapping agent A is characterized by having the ability to capture or bind amine Optionally, said amine is at least one of methylamine, dimethylamine or trimethylamine.

In some embodiment, the conjugate, plurality of conjugates or composition, device, battery and apparatus used by the therapeutic methods of the invention are any of those disclosed by the present disclosure.

In some embodiments, the conjugate used by the therapeutic methods of the present disclosure is having the structural formula V, the conjugate comprising a particle covalently bonded to at least one linker comprising a chain of 15 carbon atoms covalently bonded to m carbonyl groups, and a sulfonic acid covalently bonded to the m^{th} carbonyl group

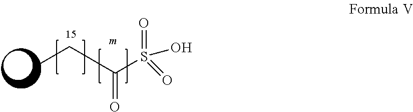

wherein m is an integer between 5 to 10.

In some embodiments, the methods of the present disclosure may be any disorders associated with elevated blood ammonia levels is a chronic hepatic or pulmonary condition and/or cognitive decline, and/or hyperammonemia and associated conditions.

In some specific embodiments, hepatic condition is hepatic encephalopathy and any related conditions.

These and other aspects of the invention will become apparent by the hand of the following disclosure.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1 is an isometric and partially broken view of a device according to an embodiment of the presently disclosed subject matter.

FIG. 2 is an isometric exploded view of the embodiment of FIG. 1.

FIG. 3A-3C. FIG. 3(a) is a side view of the outer casing of the embodiment of FIG. 1; FIG. 3(b) is a front view of the embodiment of FIG. 3(a); FIG. 3(c) is a cross-sectional side view of the embodiment of FIG. 3(b) taken along B-B.

FIG. 4A-4D. FIG. 4(a) is a side view of the inlet cap of the embodiment of FIG. 1; FIG. 4(b) is a cross-sectional side view of the embodiment of FIG. 4(a) taken along A-A; FIG. 4(c) is a back isometric view of the embodiment of FIG. 4(a); FIG. 4(d) is a front isometric view of the embodiment of FIG. 4(a).

FIG. 5A-5D. FIG. 5(a) is a side view of the outlet cap of the embodiment of FIG. 1; FIG. 5(b) is a cross-sectional side view of the embodiment of FIG. 5(a) taken along A′-A′; FIG. 5(c) is a back isometric view of the embodiment of FIG. 5(a); FIG. 5(d) is a front isometric view of the embodiment of FIG. 5(a).

FIG. 6 is a partial isometric exploded view of the first self-locking arrangement of the embodiment of FIG. 1.

FIG. 7 is a partial isometric exploded view of the second self-locking arrangement of the embodiment of FIG. 1.

FIG. 8 is a cross-sectional side view of the first barrier member assembly of the embodiment of FIG. 1.

FIG. 9 is a cross-sectional side view of the second barrier member assembly of the embodiment of FIG. 1.

FIG. 10 is a schematic illustration of a system according to an embodiment of the presently disclosed subject matter.

FIG. 11 is a schematic illustration of a system according to an alternative variation of the embodiment of FIG. 10.

FIG. 12 is a schematic illustration of a system according to another alternative variation of the embodiment of FIG. 10.

FIG. 13 is a schematic illustration of a system according to another alternative variation of the embodiment of FIG. 10.

FIG. 14 is a schematic illustration of a system according to another alternative variation of the embodiment of FIG. 10.

FIG. 15: Conjugate with Sulfonic Acid

The figure shows a schematic representation of the chemical reaction for the preparation of Conjugate with sulfonic acid.

FIG. 16: Ammonia Standard Curve

Graph representing a standard curve for calculation of ammonia concentration.

FIG. 17A-17B Establishment of Hyper Ammonia Model in Pigs

FIG. 17A shows an example of a pig that anesthetized and a central venous infusion of xylazine and ketamine.

FIG. 17B. shows a histogram showing the ammonia levels that were monitored before and after the procedure, every 30 min.

FIG. 18 Ammonia Depletion Procedure

The figure shows the procedure of depleting ammonia from a human plasma unit. The plasma bag is connected to the ammonia depleting device of the present disclosure. A flow regulator regulates the flow of the plasma into the device, and the clamp used to stop the flow in the event of leakage. The filtered blood product is than collected in a bag.

FIG. 19: Ammonia Depletion Procedure in Human Plasma

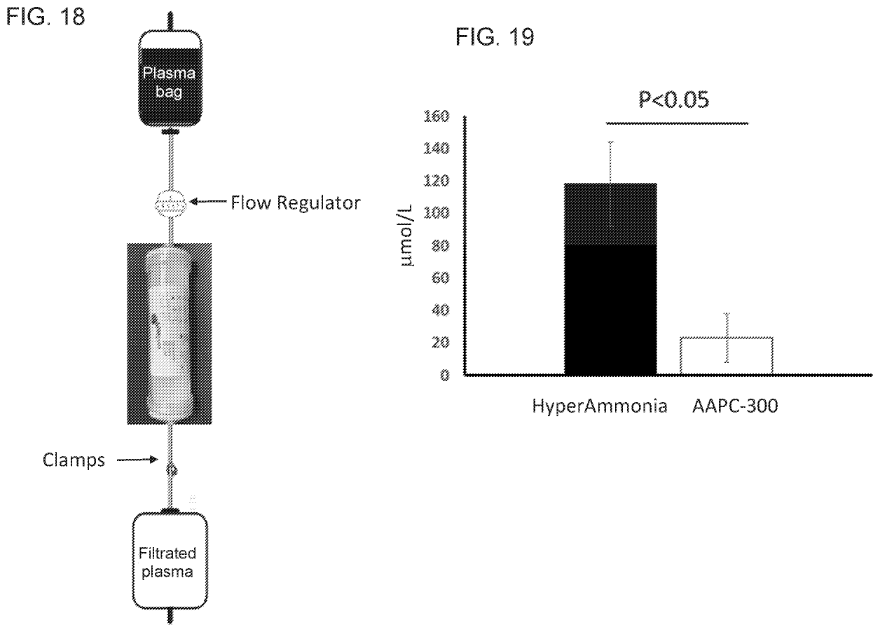

Human plasma bag (ammonia enriched) was connected to the device of the present disclosure (designated herein as AAPC-300 filter) by a Luer lock connection. Plasma flew through the device in a regulated rate of 150 mL/min (regulated by the flow regulator shown in FIG. 18). Plasma was collected in 200 mL tubes. The filtered plasma was subjected to Elisa reader, for evaluating the rate of ammonia depletion. Statistics were computed using student t-test (Two tailed distribution equal variance). Data is expressed as the Mean±SD.

### DETAILED DESCRIPTION OF THE INVENTION

The present disclosure provides, in accordance with its broadest aspect, conjugates, a plurality of conjugates, a composition comprising the plurality of conjugates, each conjugate is of a general Formula (I′)

X-Y-Z (I′)

wherein:
X is a solid support moiety, for example a particle;
Y is a chemically reactive moiety linking moieties X and Z;
Z is a moiety comprising at least one of, a trapping agent, a derivative thereof or analog thereof; and
wherein each “-” designates an interaction/association, for example a chemical bond containing optionally one or multiple intervening atoms that serve as spacers or as selectivity directing moieties. Thus, in a first aspect, the present invention provides a device comprising:
   a housing having at least one fluid inlet port, and at least one fluid outlet port;
   the housing including at least one chamber, said at least one chamber defining a control volume in fluid communication with the at least one fluid inlet port and the at least one fluid outlet port;
      said control volume accommodating at least one of a conjugates a plurality of conjugates or at least one composition comprising the conjugates or plurality of conjugates. In some specific embodiments, the conjugate of the device disclosed herein is having the structural formula I, the conjugate comprising a particle bonded to at least one linker comprising a chain of n carbon atoms covalently bonded to m carbonyl groups, and at least one trapping agent A covalently bonded to the m^{th} carbonyl group;

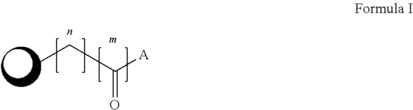

wherein, n is an integer within the range of 5 to 15, and m is an integer within the range of 5 to 10. In some embodiments, the trapping agent A is characterized by having the ability to capture or bind amine. As indicted above, in some embodiments the linker comprises 5 to 15 carbons. In some embodiments, since the length of one carbon atom is about 1.5 angstroms, thus, in some embodiments, the length of the linker may range between 7.5 or less to 22.5 or more, angstroms. In yet some further embodiments, the that in some embodiments, the linker further comprises between 5 to 10 carbonyls. As each carbonyl may be in the length of about 1.3 angstroms, the length may range between about 6.5 or less to about 13 angstroms or more.

In other words, the present disclosure provides a device comprising conjugate comprising three moieties:
particle;
a linker; and
trapping agent.

The three moieties are linked to each other in a way that the linker connects the particle and the trapping agent.

The linker of the invention generally includes 2 groups wherein the first group comprising a straight chain alkane comprising n carbon atoms and a group of m covalently bonded carbonyl groups.

In some embodiments, the straight chain alkane of the linker of the conjugate of the device of the present disclosure is saturated or unsaturated.

In some embodiments, the straight chain alkane group may be saturated, where in other embodiments the group may be unsaturated.

In embodiments where the straight chain alkane group is unsaturated, the chain may contain between 1 to 3 double bonds.

The trapping agent moiety, designated herein as A, may include any agent having the ability to “capture” or bind amine.

In the context of the herein disclosure, the term amine relates to any compound or functional group containing at least one basic nitrogen atom with at least one lone pair of electrons. Amines according to the present disclosure may include any primary, secondary, and tertiary amines having a molecular weight (MW) of between at least 17 to at most 70 Daltons.

In some embodiments, the amine is an alkylamine, dialkylamine or trialkyl amine, wherein the MW of such amines is between 17 to 70 Daltons.

In some other embodiments, the amine is selected from methylamine, dimethylamine or trimethylamine.

In a specific embodiment, the amine is ammonia.

The linker of the invention is covalently linked to the trapping agent in a way that the m^{th} carbonyl is linked via straight linkage

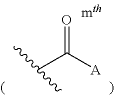

or through another short alkane chain

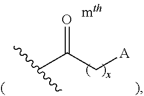

wherein X is an integer within the range of 1 to 3.

In some embodiments, the trapping agent may be any ion exchange material. More specifically, since ammonia is cationic in physiologic pH, it may bind a cation exchanger.

Other alternatives encompassed by the invention include, NHS and epoxy.

In some embodiments, the trapping agent is an acid.

In some embodiments the acid is a strong acid, capable of capturing an amine.

In some embodiments, the amine is as defined hereinabove.

In some other embodiments, the amine is ammonia.

In the context of the disclosure provided herein, the term “strong acid” is any acid having a pKa value which is lower than 1.

At times, the pKa is lower than 0, at times lower than (−1), at times lower than (−2), at times lower than (−3), at times lower than (−4), at times lower than (−5), at times lower than (−6), at times lower than (−7), at times lower than (−8), and at times lower than (−9).

In some specific embodiments, the acid is selected from the group consisting of chloric acid, hydrobromic acid, hydrochloric acid, hydroiodic acid, nitric acid, perchloric acid, sulfuric acid, hydroiodic acid, analogs thereof and derivates thereof.

In a specific embodiment, the acid is sulfuric acid or derivates thereof.

Derivates of sulfonic acid may be any molecule having the general formula:

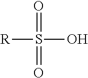

wherein R is an organic alkyl or aryl group.

In embodiments, the sulfonic acid derivate is selected from taurine, PFOS, p-Toluenesulfonic acid and coenzyme M.

In some embodiments, R is —H.

In most general terms, the length of the straight chain alkanes determines the specificity of the conjugate. A linker which is too long would capture unspecific/unwanted molecules such as proteins and amino acids (since they comprise an amino group). Short linkers would decrease the ability of the conjugate to capture ammonia and ammonium cations.

Thus, in embodiments, the length of the straight chain alkanes (n) is between 5-20 carbon atoms, specifically, 5-19, 5-18, 5-17, 5-16, 5-15, 5-14, 5-13, 5-12, 5-11, 5-10, 6-20, 7-20, 8-20, 9-20, 10-20, 1-20, 12-20, 13-20, 14-20, 15-20. In yet some further embodiments, the length of the straight chain alkanes (n) is at times, between 10-15, at times between 12-15, at times between 13-15, at time between 14-15. In some specific embodiments, the length of the straight chain alkanes (n) is 5 or less, 6, 7, 8, 9, 10, 11, 12, 13, 14, 15, 16, 17, 18, 19, 20, or more. In a specific and non-limiting embodiment, n is 15.

Without wishing to be bound by theory or mechanism, the carbonyls moiety provides the appropriate bulkiness which prevents the attachment of amino acids and peptides to the acid moiety of the conjugate. The inventors of the present disclosure surprisingly discovered that a group of between 5-10 carbonyls bonded together, provides the adequate bulkiness which prevents bigger molecules from being captured by the acidic moiety of the conjugate.

In other embodiments of the invention there is provided a device, wherein the conjugate of the disclosed device comprising a particle bonded to at least one linker comprising a chain of 15 carbon atoms covalently bonded to between 5 to 10 carbonyl groups (m), and an acid A covalently bonded to the m^{th} carbonyl group, the conjugate having the structural formula II:

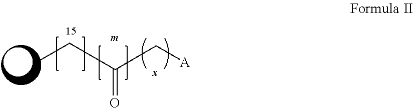

wherein x is between 0 to 3.

When x is 0, the m^{th} carbonyl is directly connected to the acid.

In some further embodiments, the invention provides device comprising a conjugate comprising a particle bonded to at least one linker comprising a chain of 15 carbon atoms covalently bonded to between 5 to 10 carbonyl groups (m), and a sulfonic acid covalently bonded to the m^{th} carbonyl group, the conjugate having the structural formula III:

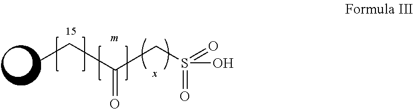

wherein x is between 0 to 3.

In some embodiments, x is between 0 to 2, at times between 0 to 1. At times x is 1, and at times x is 0.

When x is 0, the m^{th} carbonyl is directly connected to the acid.

In some embodiments, the conjugate of the device of the present disclosure is configured to act as an amine trap, exhibiting a neutralization reaction of amines. In more specific embodiments, the amine is ammonium.

Still further, in some embodiments, the linker moiety of the conjugate of the present disclosure is connected to the particle via any suitable functional group allowing connecting the particle to the straight chain alkane.

Without being bound by theory or reaction mechanism, conjugates of the invention act as an amine trap exhibiting a neutralization reaction of one amine as defined above for each acid trapping agent, in the following manner

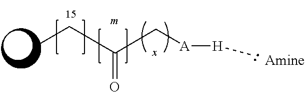

wherein —H is the free hydrogen atom of the acid and the (:) are the free electrons of the amine.

In embodiments where the acid is sulfonic acid and the amine is ammonia, a proton (H⁺) is donated to the two free electrons of ammonia as follows:

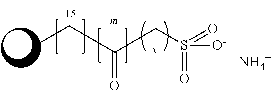

generating an ionic bond between the conjugate acid and conjugate base.

Linker moiety of the herein disclosure may be connected to the particle via any group known in the art, which allows connecting the particle to the straight chain alkane.

In one possible embodiment, the particle and the linker and covalently connected.

In another optional embodiments, the linkage is a covalent linkage via amino group as presented in Formula IV:

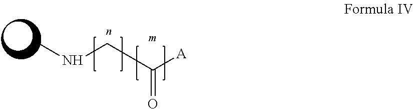

In yet some other embodiments, the invention contemplates devices that comprise at least one conjugate having the structural formula V, wherein n is 15, m is an integer between 5 to 10, x is 0 and A is a sulfonic acid:

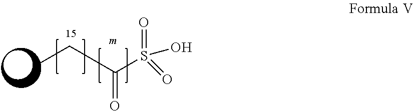

The preset invention provides at least one device comprising at least one conjugate. A conjugate as used herein refers to a compound constructed from several elements (components), including at least one particle, at least one linker and at least one trapping agent, specifically a trapping agent for ammonia, that may be in some embodiments a sulfonic acid or any derivative thereof or an analog thereof which are all associated thereto. It should be noted that while the application refers to a “at least one particle”, any solid support being applicable for the claimed plurality of conjugate is encompassed herein.

Any one of the conjugates of the presently disclosed devices of the present invention, or any compositions thereof, may also be referred to as a composition of matter. In most general terms, a “composition of matter” similarly to a “conjugate”, both used interchangeably, refers to the association of the at least one particle, the at least one linker and the at least one trapping agent, derivative thereof or analog thereof, that as detailed below produces properties which may be attributed to the composition of matter (or conjugate) as a whole and not to any one of conjugate's components in their separate state.

In some embodiments, any one of the conjugates of the presently disclosed devices encompasses an association of the at least one particle with the at least one chemically reactive moiety being a linker and the association of the at least one linker with the at least one trapping agent, specifically, sulfonic acid and derivative thereof or an analog thereof such that the linker is positioned between the particle and the trapping agent, derivative thereof or an analog thereof and hence being associated at one end (at one arm) to the particle and at another end (at a second different arm) to the trapping agent, derivative thereof or an analog thereof.

In some embodiments, a trapping agent, specifically, sulfonic acid and derivative thereof, binds specifically and selectively to a particular target, in this case at least one amine, for example, ammonia and enables effective trapping, immobilization, partitioning and removal of the ammonia from the liquid substance, specifically, body fluid.

As used herein, the term “association” or any linguistic variation thereof refers to the chemical or physical force which holds two entities together (e.g., the particle and the linker). Such force may be any type of chemical or physical bonding interaction known to a person skilled in the art.

Non-limiting examples of such association interactions are covalent bonding, ionic bonding, coordination bonding, complexation, hydrogen bonding, van der Waals bonding, hydrophobicity-hydrophilicity interactions, etc. Thus, the association/conjugation of the linker with the at least one particle and of the linker with the trapping agent may be via any chemical bonding, including covalent bonding, electrostatic interaction, acid base interaction, van der Waals interaction, etc. As appreciated, the association of the particle and the linker and the association of the linker with the trapping agent, a derivative thereof or an analog thereof may be the same or may be different as further detailed below.

Particles of the invention may include any polymeric particle which is able to bind the linker of the invention.

In some embodiments, the particle of the disclosed device is a resin bead.

The term particles as used herein refers to a portion of matter having a surface that can be attached to chemical or biological compounds, small or large molecules that may be attached through either covalent or non-covalent bonds. The particle may comprise a porous material. The particles may be “spherical” (refers generally to a substantially (nearly) round-ball geometry) or “non-spherical”, for example, (“elongated” in shape and has a defined long and short axis). Non-liming examples of particles include beads such as at least one of polysaccharide bead, glass beads, cotton bead, plastic bead, nylon bead, latex bead, magnetic bead, paramagnetic bead, super paramagnetic bead, starch bead and the like, silicon bead, PTFE bead, polystyrene bead, gallium arsenide bead, gold bead, or silver bead. In some embodiments, the particle is a bead comprising agarose beads, optionally at different degree of crosslinking at different % of material (agarose).

As such, agarose beads encompass beads comprising agarose at varying degree of crosslinking, for example beads denoted as Sepharose beads. In some embodiments, the bead comprises agarose beads. In some embodiments, the bead comprises Sepharose beads. In some embodiments, the plurality of conjugates comprises a combination of particles comprising agarose beads and Sepharose beads. In accordance with the present disclosure, it should be noted that particles being either agarose beads and Sepharose beads are considered as two different conjugates having different particles.

Sepharose is a tradename for a crosslinked, beaded-form of agarose, a polysaccharide polymer material extracted from seaweed. Its brand name is derived from Separation-Pharmacia-Agarose. Sepharose is a registered trademark of GE Healthcare (formerly: Pharmacia, Pharmacia LKB Biotechnology, Pharmacia Biotech, Amersham Pharmacia Biotech, and Amersham Biosciences). Various grades and chemistries of Sepharose are available.

The particle and specifically the bead of the devices as described herein may be associated to a chemically reactive moiety, denoted herein as a linker. The linker as used herein may be any chemical entity that is composed of any assembly of atoms, including oligomeric and polymeric chains of any length, which according to some embodiments, is capable of binding on one end to the particle and on the other end the at least one trapping agent, a derivative thereof or an analog thereof.

In some embodiments, the bead may be associated to the linker via a spacer or coating present on the bead. As such, the bead is initially activated (“activated beads”) by association to a spacer/coating and then reacted with a linker. It should be noted that at times, no linker may be further required in cases the spacer/coating binds directly to the at least one trapping agent. At times, the bead does not have a functional group capable of binding to the linker, and a spacer or coating may be used.

The activated beads are obtained by pre-coating the beads with a suitable material having an active moiety enabling the binding to the beads and to the linker and/or the trapping agent. In other words, the beads are pre-coated to include reactive groups enabling the covalent binding to either the linker or the trapping agent.

In some embodiments the beads of the conjugates of the disclosed devices, may be activated for example by pre-coating with any coating material. Non-limiting examples of such material include for example, amino acid, protein, epoxy, tosyl, carboxylic acid, carboxylated polyvinyl alcohol. when referring to “pre-coating” it should be understood as a preliminary step which results in coating of the beads with an active material that in turn enables covalent binding of the beads with the sulfonic acid (i.e., directly) or via at least one linker. In some embodiments, the beads of the conjugates of the disclosed devices, are pre-coated with an amino acid, peptide or any derivative thereof. Pre-coated magnetic beads may comprise for example as active groups, a primary amine (—NH2), carboxyl (—COOH), sulfhydryl (—SH) or carbonyl (—CHO). In some embodiments, the beads of the conjugates of the disclosed devices, are pre-coated to include a moiety that may react with primary or secondary amino groups. In some other embodiments, the magnetic beads are coated with polylysine.

As used herein the term “linker” encompasses any spacer or pre-coating present on the beads.

In some embodiments, the linker comprises or is a chain of atoms, for example a linear chain. In some embodiments, the linker comprises at least 1 atom, at least 4 atoms, at times 5 atoms, at times 10 atoms, at times 20 atoms, at times 30 atoms, at times 40 atoms. In some embodiments the linker is or comprises a linear chain of 1 to 40 atoms. In some embodiments the linker is or comprises a linear chain of 1 atom. In some embodiments the linker is a linear chain comprising 5 atoms. In some embodiments the linker is a linear chain comprising 15 atoms.

In some embodiments, the particle is a resin bead. In some embodiments, the particle may be an agarose bead, and therefore, the resin bead is in some embodiments the agarose resin may comprise between about 2% to 10% agarose. Still further, in some embodiments the resin bead may comprise between 3% to 9% agarose, still further in some embodiments, the resin bead may comprise between 4% to 8% agarose. According to further specific any non-limiting embodiments, the resin bead comprising at least 4% of agarose. In some other embodiments, the amount of agarose in the particle of the conjugates of the disclosed devices, is at least 5%, and at times at least 6%.

In some embodiments, the conjugate of the devices of the resent disclosure comprises particles having an average particle size of between about 10 μm or less, to about 500 μm or more. Specifically, 10μm, 20 μm, 30 μm, 40 μm, 50 μm, 60 μm, 70 μm, 80 μm, 90 μm, 100 μm, 110 μm, 120 μm, 130 μm, 140 μm, 150 μm, 160 μm, 170 μm, 180 μm, 190 μm, 200 μm, 250 μm, 300 μm, 350 μm, 400 μm, 450 μm, 500 μm or more. In some specific embodiments, the plurality of conjugates comprise particles having an average particle size of at least 70 μm or less, at times at least 80 μm, at times at least 90 μm, at times at least 100 μm, at times at least 110 μm, at times at least 120 μm, at times at least 130 μm, at times at least 140 μm, at times at least 150 μm. In some embodiments, the plurality of conjugates of the disclosed devices, display an average particle size of between about 40 μm or less, to about 170 μm or more.

The term “average size” or “average diameter” or “mean size” refers to the arithmetic mean of measured diameters, wherein the diameters range ±25% of the mean. The mean size of the particles can be measured by any method known in the art. In certain embodiments, the size of the resin bead is between 40-170 μm, where the average size is between about 80 to about 100 μm. At times, the average size is 90 μm.

As indicated above, in accordance with the present aspect, the present invention relates to a device and system, particularly for use in enabling depleting ammonia from biological fluids.

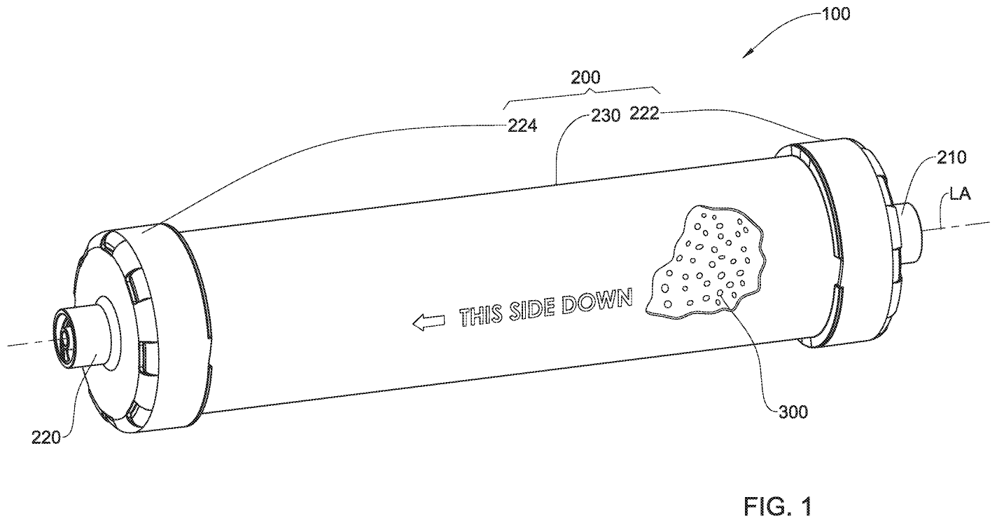

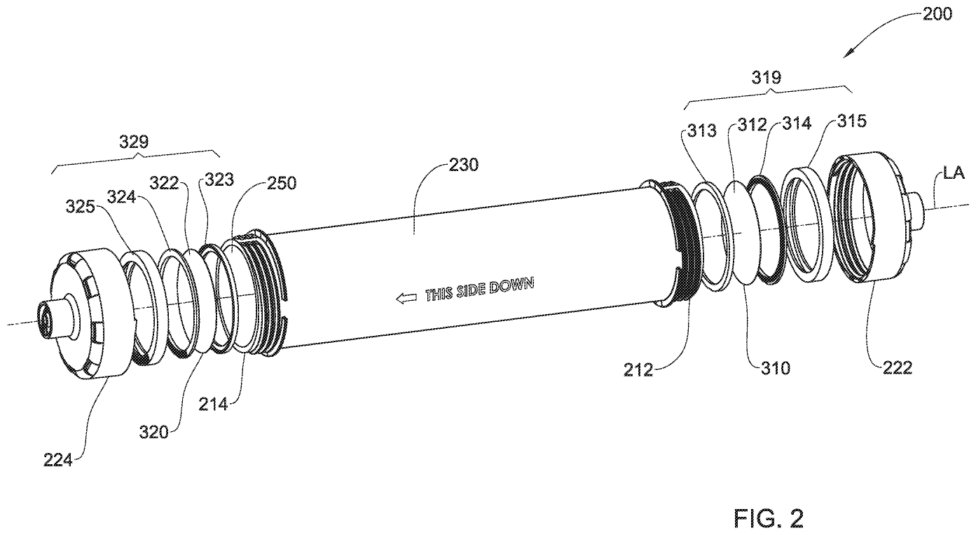

Referring to FIGS. 1 and 2, such a device according to a first embodiment of the presently disclosed subject matter, generally designated 100, comprises a housing 200 and an active material 300 accommodated therein.

The housing 200 comprises an outer casing 230, an inlet end cap 222 and an outlet end cap 224. The housing 200 defines a longitudinal axis LA.

The outer casing 230 extends longitudinally between an inlet end 212 and an outlet end 214. In at least this example the casing is generally cylindrical.

A chamber 250 is defined between the outer casing 230, the inlet end 212 and the outlet end 214, and the chamber 250 provides a control volume CV fillable with the active material 300.

In at least this example, the inlet end cap 222 is configured for being sealingly mounted to the inlet end 212, and the outlet end cap 224 is configured for being sealingly mounted to the outlet end 214.

In at least this example, the inlet end cap 222, the outlet end cap 224, and the housing 230 are each made from a suitable medically compatible materials, for example Terlux HD 2802 provided by Ineos, or Makrolon 2458 provided by Covestro.

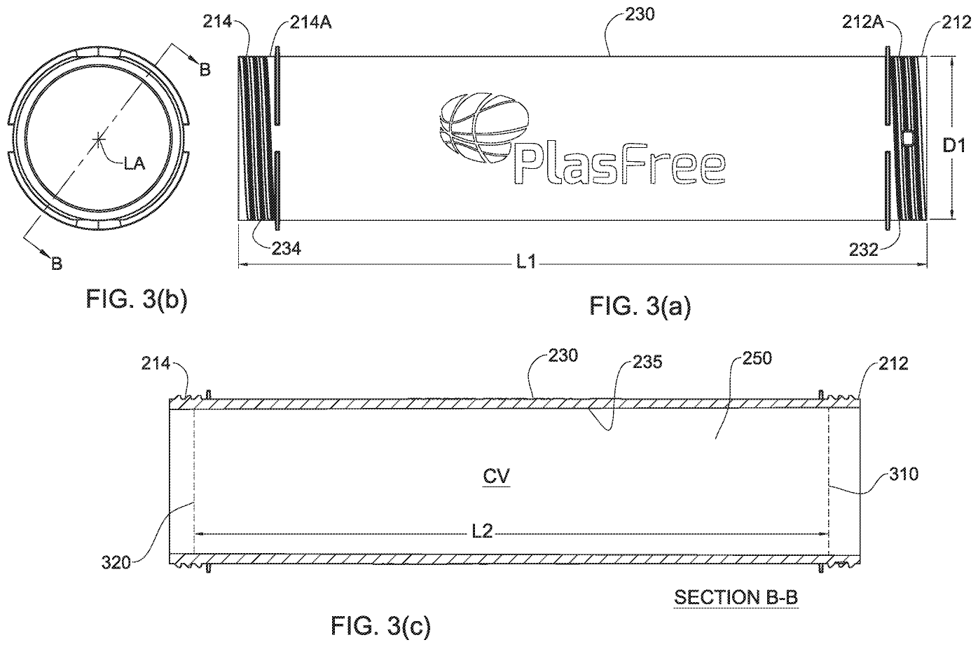

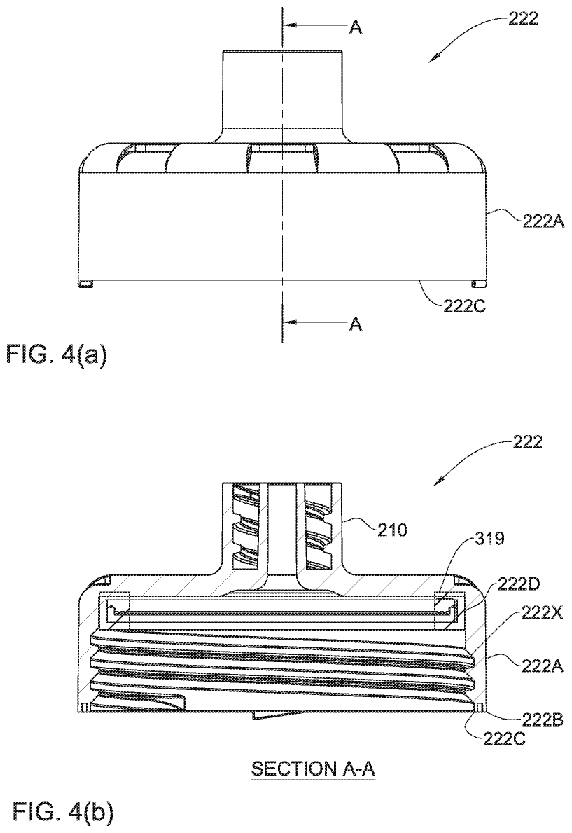

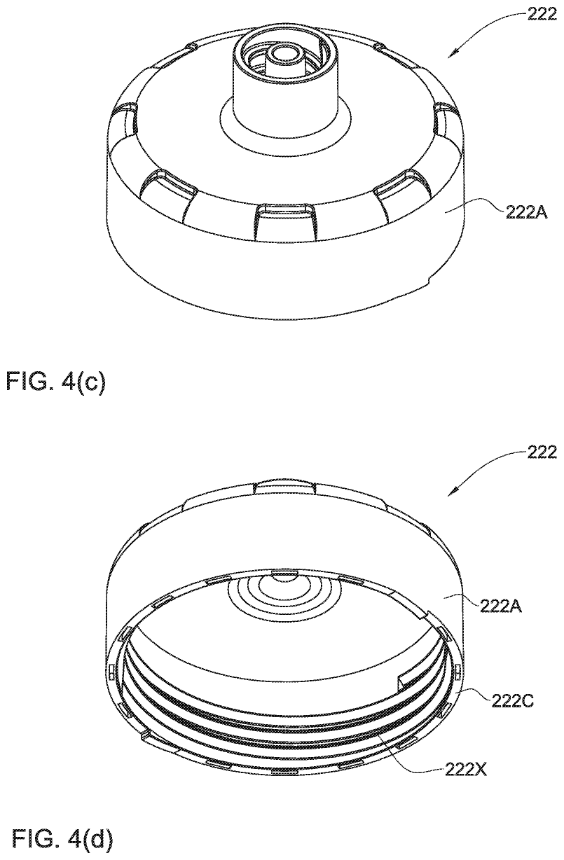

In at least this example, and referring in particular to FIGS. 3(a), 4(a) to 4(d), the inlet end cap 222 comprises a respective enlarged portion 222A, having an internal diameter sufficient for enabling the respective enlarged portion 222A to be engaged in overlying relationship with a respective engagement portion 212A at the inlet end 212. The free end 222B of the respective enlarged portion 222A comprises a generally annular flat surface 222C.

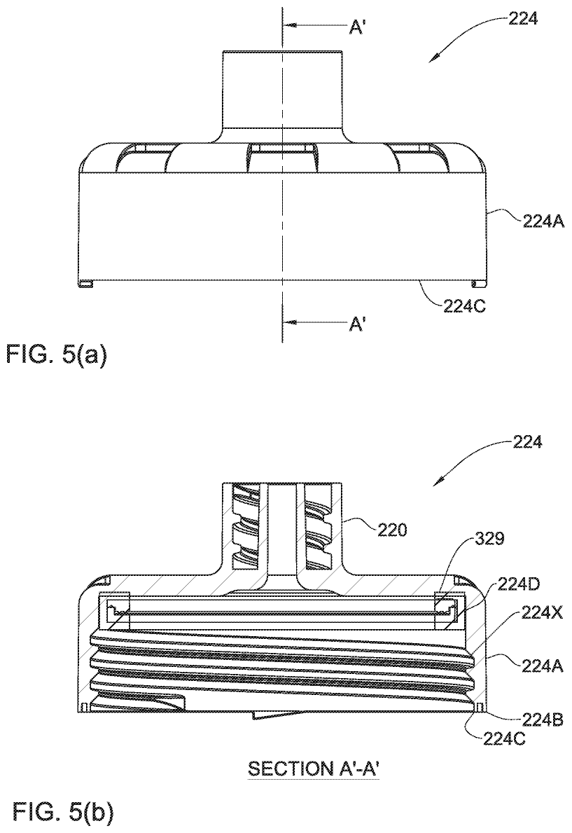

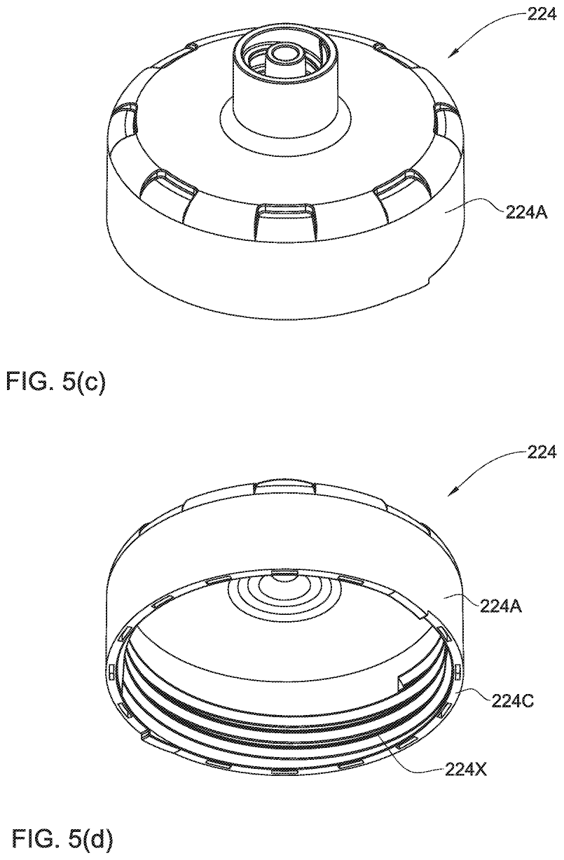

Similarly, in at least this example, and referring in particular to FIGS. 3(a), 5(a) to 5(d), the outlet end cap 224 comprises a respective enlarged portion 224A, having an internal diameter sufficient for enabling the respective enlarged portion 224A to be engaged in overlying relationship with a respective engagement portion 214A at the outlet end 214. The free end 224B of the respective enlarged portion 224A comprises a generally annular flat surface 224C.

In at least this example, and referring in particular to FIGS. 3(a), 4(b), 4(d), 5(b) and 5(d), the inlet end cap 222 and the outlet end cap 224 each comprises a respective internally threaded wall 222X, 224X, respectively, that is complementary to a respective externally threaded wall 232, 234 provided at inlet end 212 and at outlet end 214, respectively. Optionally, an external sealing tape and/or an internal O-ring (not shown) can be provided for additional sealing between the respective inlet end cap 222 and/or the outlet end cap 224, and the housing 230.

Furthermore, in at least this example inlet end cap 222 and the outlet end cap 224 are each configured as self-locking end caps with respect to the outer casing 230, enabling the respective inlet end cap 222 and/or the outlet end cap 224 to be sealingly locked in place with respect to the casing 230.

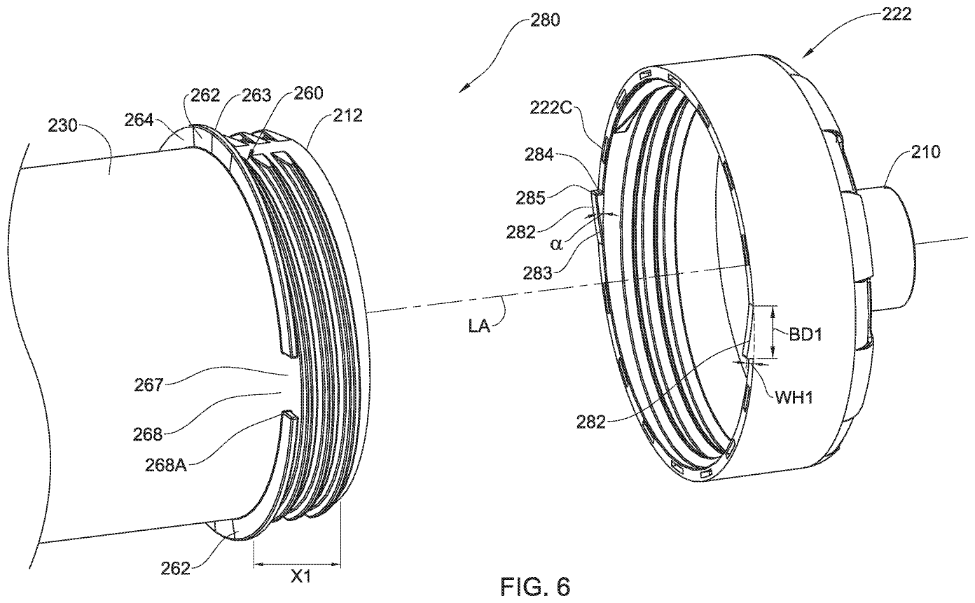

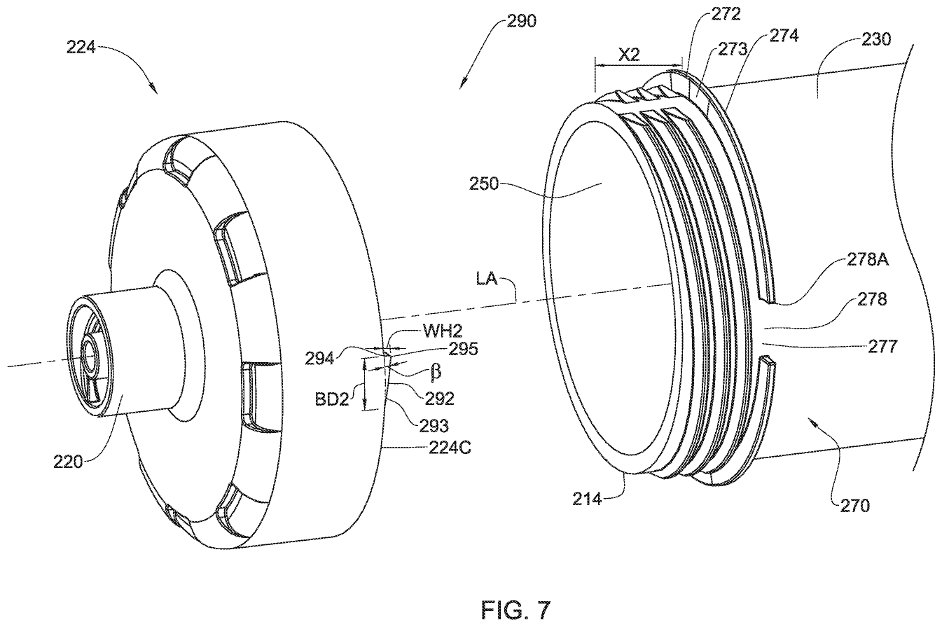

For this purpose, in at least this example, and referring also to FIGS. 6 and 7, the device 100, in particular the housing 200, comprises a first self-locking arrangement 280 configured for enabling self-locking of the inlet end cap 222 with respect to the outer casing 230, and a second self-locking arrangement 290 configured for enabling self-locking of the outlet end cap 224 with respect to the outer casing 230

Referring again to FIG. 6, the first self-locking arrangement 280 comprises a plurality of first wedge elements 282 provided in the inlet end cap 222 that cooperates with a first flange stop arrangement 260 to provide self-locking of the inlet end cap 222 with respect to the outer casing 230. While in this embodiment the inlet end cap 222 comprises two first wedge elements 282, in alternative variations of this example, the inlet end cap 222 can include one or more than two first wedge elements.

Each first wedge element 282 is projecting in a longitudinal direction away from the respective annular flat surface 222C, and furthermore the first wedge elements 282 are equi-spaced from one another in a circumferential direction along the respective annular flat surface 222C.

In at least this example, each first wedge element 282 is in the form of a right angled wedge, and comprises a respective first wedge edge 283 and a respective second wedge edge 284 meeting at a respective wedge apex 285. The wedge apex 285 is at first wedge height WH1 with respect to the annular flat surface 222C. Each first wedge element also has first base dimension BD1 at the annular flat surface 222C.

The first wedge edge 283 is sloped at an acute angle α with respect to the respective annular flat surface 222C. In at least this example, angle α is significantly less than 90°, for example in the range of between about 5° and about 30°, for example about 20°.

The second wedge edge 284 is sloped generally orthogonally with respect to the respective annular flat surface 222C. As will become clearer herein, the second wedge edge 284 cooperates with the first flange stop arrangement 260 for enabling self-locking the inlet end cap 222 with respect to the outer casing 230.

The first flange stop arrangement 260 comprises a first annular flange 262 provided on an outer surface 232 of the outer casing 230, and longitudinally spaced from the inlet end 212 by a first spacing X1. The first annular flange has a respective first annular face 263 and an opposite facing respective second annular face 264. The first annular face 263 facing a direction towards the inlet end 212, and thus the first annular face 263 is spaced from the inlet end 212 by the first spacing X1.

The first spacing X1 is sufficient such as to ensure that when the inlet end cap 222 is fully engaged (in at least this example, by screwing the inlet end cap 222 with respect to the outer casing 230) with the outer casing 230, the respective annular flat surface 222C at the respective free end 222B is in abutting contact with the annular face 263.

The first flange stop arrangement 260 further comprises a plurality of first stop elements 268 corresponding to the plurality of first wedge elements 282. Thus, in at least this example, the first flange stop arrangement 260 comprises two first stop elements 268 corresponding to the two first wedge elements 282.

In at least this embodiment, each first stop element 268 is in the form of a respective abutment surface 268A provided in the first annular flange 262. The first annular flange 262 thus comprises cutouts 267 corresponding to the first wedge elements 282, and each cutout 267 has a circumferential length and an axial depth at least equal to the first base dimension BD1 and first wedge height WH1, respectively, of each first wedge element 282 to enable accommodating the respective first wedge element 282 therein, and a respective abutment surface 268A for abutting with respect to the respective second wedge edge 284.

Thus, as the inlet end cap 222 is screwed onto the first inlet end 212 of the outer casing 230 in an engagement rotational direction, eventually the first wedge elements 282 abut the first annular face 263. As the inlet end cap 222 is screwed further onto the first inlet end 212 of the outer casing 230, the first wedge elements 282 and/or the parts of the first annular flange 262 in contact therewith deform slightly until the respective the first wedge elements 282 snap into the respective cutouts 267 via the respective first wedge edges 283. Thereafter, the respective second wedge edges 284 are in abutting contact with the respective abutment surfaces 268A preventing relative rotation between the inlet end cap 222 and the outer casing 230 in a disengagement direction.

In alternative variations of this embodiment, the first flange stop arrangement 260 can be in a different form—for example the first flange 262 can be replaced with a plurality of mechanical stop, for example each comprising a boss projecting radially from the outer casing 230. Each such mechanical stop corresponds to a different one of said first wedges 282, and is located on an outer surface of the outer casing 230 at a respective location corresponding to the location of the respective first wedge element 282 when the inlet cap 222 is fully screwed into place. Each such boss comprises a respective abutment surface for abutting with respect to the respective second wedge edge 284.

Referring again to FIG. 7, the second self-locking arrangement 290 comprises a plurality of second wedge elements 292 provided in the outlet end cap 224 that cooperates with a second flange stop arrangement 270 to provide self-locking of the outlet end cap 224 with respect to the outer casing 230. While in this embodiment the outlet end cap 224 comprises two second wedge elements 292, in alternative variations of this example, the outlet end cap 224 can include one or more than two second wedge elements.

Each second wedge element 292 is projecting in a longitudinal direction away from the respective annular flat surface 224C, and furthermore the second wedge elements 284 are equi-spaced from one another in a circumferential direction along the respective annular flat surface 224C.

In at least this example, each second wedge element 292 is in the form of a right angled wedge and comprises a respective first wedge edge 293 and a respective second wedge edge 294 meeting at a respective wedge apex 295. The wedge apex 295 is at second wedge height WH2 with respect to the annular flat surface 224C. Each second wedge element 292 also has second base dimension BD2 at the annular flat surface 224C.

The respective first wedge edge 293 is sloped at an acute angle R with respect to the respective annular flat surface 224C. In at least this example, angle R is significantly less than 90°, for example in the range of between about 5° and about 30°, for example about 20°.

The respective second wedge edge 294 is sloped generally orthogonally with respect to the respective annular flat surface 224C. As will become clearer herein, the second wedge edge 294 cooperates with the second flange stop arrangement 270 for enabling self-locking the outlet end cap 224 with respect to the outer casing 230.

The second flange stop arrangement 270 comprises a respective first annular flange 272 provided on the outer surface 232 of the outer casing 230, and longitudinally spaced from the outlet end 214 by a second spacing X2. The second annular flange 272 has a respective first annular face 273 and an opposite facing respective second annular face 274. The first annular face 273 is facing a direction towards the outlet end 214, and thus the respective first annular face 273 is spaced from the outlet end 214 by the second spacing X2.

The second spacing X2 is sufficient such as to ensure that when the outlet end cap 224 is fully engaged (in at least this example, by screwing the outlet end cap 224 with respect to the outer casing 230) with the outer casing 230, the respective annular flat surface 224C at the respective free end 224B is in abutting contact with the annular face 273.

The second flange stop arrangement 270 further comprises a plurality of second stop elements 278 corresponding to the plurality of second wedge elements 292. Thus in at least this example, the second flange stop arrangement 270 comprises two second stop elements 278 corresponding to the two second wedge elements 292.

In at least this embodiment, each second stop element 278 is in the form of a respective abutment surface 278A provided in the second annular flange 272. The second annular flange 272 thus comprises cutouts 277 corresponding to the second wedge elements 292, and each cutout 277 has a circumferential length and an axial depth at least equal to the second base dimension BD2 and second wedge height WH2, respectively, of each second wedge element 292, to enable accommodating the respective second wedge element 292 therein, and a respective abutment surface 278A for abutting with respect to the respective second wedge edge 294.

Thus, as the outlet end cap 224 is screwed onto the outlet end 214 of the outer casing 230 in an engagement rotational direction, eventually the second wedge elements 292 abut the respective first annular face 273. As the outlet end cap 224 is screwed further onto the outlet end 214 of the outer casing 230, the second wedge elements 292 and/or the parts of the second annular flange 272 in contact therewith deform slightly until the respective the second wedge elements 292 snap into the respective cutouts 277 via the respective first wedge edges 293. Thereafter, the respective second wedge edges 294 are in abutting contact with the respective abutment surfaces 278A preventing relative rotation between the outlet end cap 224 and the outer casing 230 in a disengagement direction.

In alternative variations of this embodiment, the second flange stop arrangement 270 can be in a different form—for example the second annular flange 272 can be replaced with a plurality of mechanical stops, for example each comprising a boss projecting radially from the outer casing 230. Each such mechanical stop corresponds to a different one of said second wedge elements 292, and is located on an outer surface of the outer casing 230 at a respective location corresponding to the location of the respective second wedge element 292 when the outlet cap 224 is fully screwed into place. Each such boss comprises a respective abutment surface for abutting with respect to the respective second wedge edge 294.

However, in alternative variations of this example, different configurations can be provided for sealingly mounting the inlet end cap 222 and/or the outlet end cap 224 to the casing 230.

Thus, the inlet end cap 222 and/or the outlet end cap 224 according to at least this embodiment facilitate the process of filling the control volume CV with active material 300. One of the inlet end cap 222 and the outlet end cap 224 is sealingly mounted to the casing 230, leaving open the other outlet end 214 or inlet end 212, respectively. The chamber 250 can then be filled with the desired quantity of active material 300, via the open outlet end 214 or inlet end 212. Thereafter, the chamber 250 can be closed by sealingly mounting the outlet end cap 224 or the inlet end cap 222 to the open outlet end 214 or inlet end 212, respectively, and this can be dome in a simple manner, typically manually and not requiring special equipment, or not disturbing or damaging the active material 300 in the control volume CV.

Referring in particular to FIG. 1, in at least this embodiment, the device 100 comprises a fluid inlet port 210, a fluid outlet port 220. However, in alternative variations of this embodiment, the respective device can have more than one fluid inlet ports and/or more than one fluid outlet ports. In any case, the fluid inlet port 210 and the fluid outlet port 220 are in fluid communication with the chamber

In at least this example, the fluid inlet port 210 is provided in the inlet end cap 222, and the fluid outlet port 220 is provided in the outlet end cap 224.

Referring again to FIG. 2, in at least this example, the device further comprises a first barrier member 310 at the inlet end 212, and a second barrier member 320 at the outlet end 214.

Each one of the first barrier member 310 and the second barrier member 320 is configured for permitting fluid flow, in particular liquid plasma flow in one direction through the respective barrier member, but blocking fluid flow, in particular liquid plasma flow in the opposite direction through the respective barrier member, Thus, each one of the first barrier member 310 and the second barrier member 320 operates as a respective one-way valve.

The first barrier member 310 and the second barrier member 320 are oriented in the device 100 such as to permit fluid flow, in particular liquid plasma flow, in a direction through the device 100 from the fluid inlet port 210 to the fluid outlet port 220, and concurrently blocking reverse flow through the device 100 from the fluid outlet port 220 to the fluid inlet port 210.

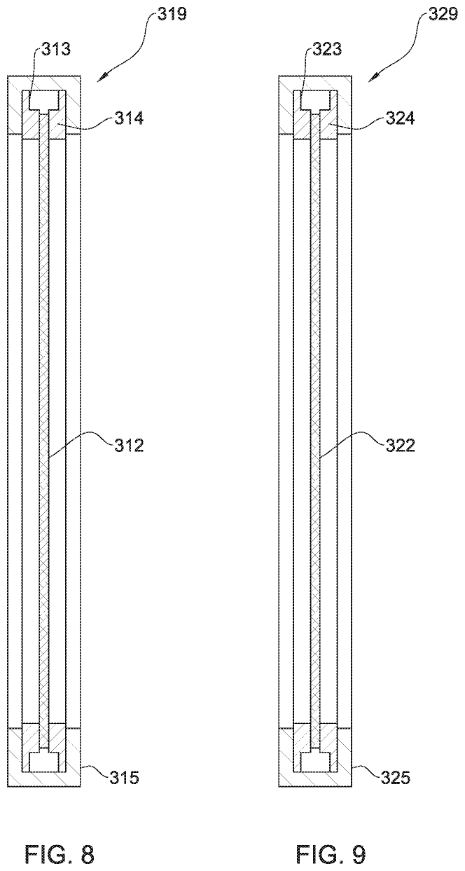

Referring in particular to FIG. 2 and FIG. 8, in at least this example the first barrier member 310 in a provided in a first barrier member assembly 319, and comprises a membrane member 312 in the form of disc, and made from a suitable medically compatible material, for example polyethersulfone (PES) material, having pore size of for example 15 μm, thickness of for example 145.7 μm, diameter about 44.5 mm. In the first barrier member assembly 319, the membrane member 312 is fixedly clamped between a respective first ring 313 and a respective second ring 314, and accommodated in an annular gasket member 315, which has a U-shaped cross section. Referring also to FIG. 4(b), the enlarged portion 222A includes in internal shoulder 222D, located between the respective internally threaded wall 222X and the fluid inlet port 210, in which the first barrier member assembly 319 is mounted.

Similarly, and referring in particular to FIG. 2 and FIG. 9, in at least this example the second barrier member 320 in a provided in a second barrier member assembly 329, and comprises a membrane member 322 in the form of disc, and from a suitable medically compatible material, for example, polyethersulfone (PES) material, having pore size of for example 15 μm, thickness of for example 145.7 μm, diameter about 44.5 mm. In the second barrier member assembly 329, the membrane member 322 is fixedly clamped between a respective first ring 323 and a respective second ring 324, and accommodated in an annular gasket member 325, which has a U-shaped cross section. Referring also to FIG. 5(b), the enlarged portion 224A includes in internal shoulder 224D, located between the respective internally threaded wall 224X and the fluid outlet port 220, in which the second barrier member assembly 329 is mounted.

In alternative variations of this and other examples, the barrier members can include, for example, filters comprising fibers or plastic substrates wherein the ligand is conjugated to the fibers or plastic substrates, in which the ligand is conjugated, or for example other suitable membranes, for example one-way membranes that allow flow there though of body fluids in one direction but not in the opposite direction through the membrane.

Referring in particular to FIG. 3(c), the control volume CV is enclosed and bound by the first barrier member 310, the second barrier member 320 and the inner surface 235 of the housing outer casing 230.

In at least this example, and referring to FIG. 3(a) in particular, the housing 200 has a longitudinal length L1 of between about 200 mm and about 210 mm, for example 205 mm. In at least this example, the housing 200 has an external dimeter D1 of between about and about 50 mm, for example 48.8 mm.

Referring to FIG. 3(c), in at least this example the chamber 250, and in particular the control volume CV, has a longitudinal length L2 of between about 200 mm and about 210 mm, for example 205 mm, and a diameter D2 of between about 40 mm and about for example 42.8 mm.

In at least this example the chamber 250 having control volume CV of between about 257 ml and 326 ml, for example, 306 ml which to accommodate the active material 300.

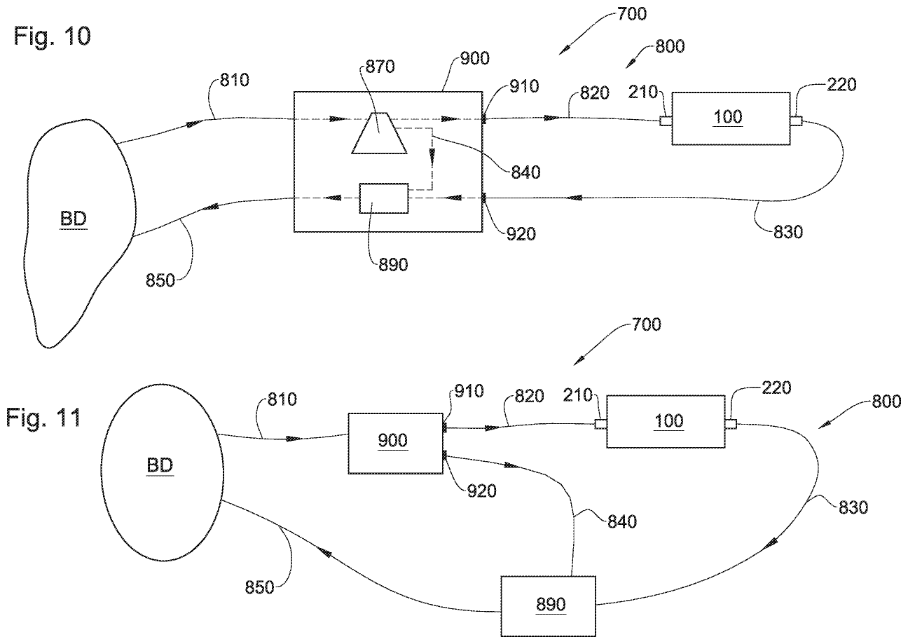

According to this aspect of the presently disclosed subject matter, and referring to FIG. 10, there is also provided a system 700 for enabling depleting ammonia from biological fluids, comprising at least one device 100, an apheresis machine 900 (including a pool or blood mixing reservoir 890, and a separating system 870, for example including a centrifuge and pumps), and a conduit system 800.

The apheresis machine 900 is configured for receiving blood from a body of a subject in need thereof, referred to herein as BD, via the conduit system 800, and for separating the received blood into its various components: plasma, platelets, white blood cells and red blood cells. Many examples of commercially available apheresis machines are known, for example the Spectra Optia Apheresis System by Terumo BCT. The apheresis machine 900 is also configured for returning treated blood to the body of a subject in need thereof, BD, via the conduit system 800, after treatment via the device 100.

The blood mixing reservoir 890, which at least in this example is integral with the apheresis machine 900, is configured for receiving plasma treated by the device 100, and blood products separated from plasma by separating system 870 of the apheresis machine 900 (for example platelets, white blood cells and red blood cells and some original plasma). The blood mixing reservoir 890 is also configured for enabling mixing of the received treated plasma and blood products to provide a treated blood.

The conduit system 800 comprises a first conduit 810 providing selective fluid communication between the apheresis machine 900 and the body of a subject in need thereof, BD, and enables blood to flow to the apheresis machine 900 from the body of a subject in need thereof, BD.

The conduit system 800 comprises a second conduit 820 providing fluid communication from the plasma outlet 910 of the apheresis machine 900 and the fluid inlet port 210 of the device 100, enabling plasma, separated from blood by the apheresis machine 900, to flow into the device 100.

The conduit system 800 comprises a third conduit 830 providing fluid communication from the fluid outlet port 220 of the device 100 to a blood mixing reservoir 890 of the apheresis machine 900 via port 920, enabling processed plasma, treated by the device 100, to flow into the blood mixing reservoir 890.

The conduit system 800 comprises a fourth conduit 840 (within the apheresis machine 900) providing fluid communication from the blood products outlet of the separation system 870 of the apheresis machine 900 to the blood mixing reservoir 890, enabling other products separated from plasma by the separating system 870 of the apheresis machine 900, to flow into the blood mixing reservoir 890.

The conduit system 800 comprises a fifth conduit 850 providing selective fluid communication between the blood mixing reservoir 890 of the apheresis machine 900, and the body of a subject in need thereof, BD, and enables treated blood to flow from the blood mixing reservoir 890 of the apheresis machine 900 to the body of a subject in need thereof BD.

Thus, in operation of the system 700, the system 700 is coupled to the body of a subject in need thereof, BD, via the conduit system 800, in particular via the first conduit 810 and the fifth conduit 850.

A suitable priming procedure is used for priming the system 700 with suitable fluid, for example a saline solution, prior to coupling the system 700 to the body of a subject in need thereof, BD.

The apheresis machine 900 is operated to separate incoming blood from the body of a subject in need thereof, BD, into plasma and blood products, the plasma being channeled to the device 100 via the second conduit 820, while the other blood products separated by the apheresis machine 900 are channeled to the blood mixing reservoir 890 thereof via the fourth conduit 840.

The plasma is treated in the device 100, and the treated plasma is channeled into the blood mixing reservoir 890 of the apheresis machine 900 via the third conduit 830.

Thereafter, treated blood is channeled from the blood mixing reservoir 890 of the apheresis machine 900 to the body of a subject in need thereof via the fifth conduit 850.

In alternative variations of this embodiment, and referring for example to FIG. 11, the blood mixing reservoir 890 is separate from the apheresis machine 900, and the blood mixing reservoir 890 is connected to the apheresis machine 900, device 100, and body BD via separate fourth conduit 840, third conduit 830 and fifth conduit 850, respectively.

It is to be noted that in alternative variations of these embodiment, the respective system 700 can include a plurality of such devices 100, for example a battery of such devices 100, coupled to the respective apheresis machine 900, blood mixing reservoir 890, and conduit system 800.

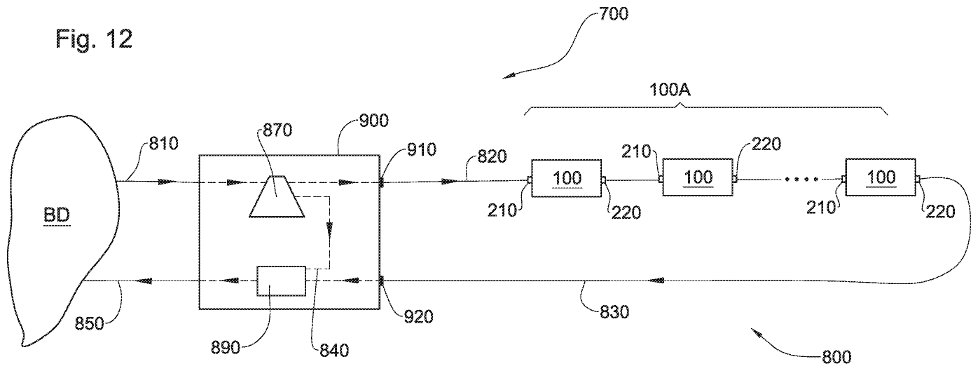

For example, in some such examples, and referring to FIG. 12, the devices 100 in such a plurality (for example in a battery 100A of such devices) can be connected with respect to one another in series, such that the fluid outlet port 220 of the first device is coupled to the fluid inlet port 210 of the next device 100 (directly or via tubing), and similar couplings are provided along the serially arranged devices 100, wherein the fluid outlet port 220 of the last device 100 is coupled to the blood mixing reservoir 890 via the respective third conduit 830. Such an arrangement can provide further treatment to the plasma before returning the treated plasm to the to the body of a subject in need thereof via the respective fifth conduit 850.

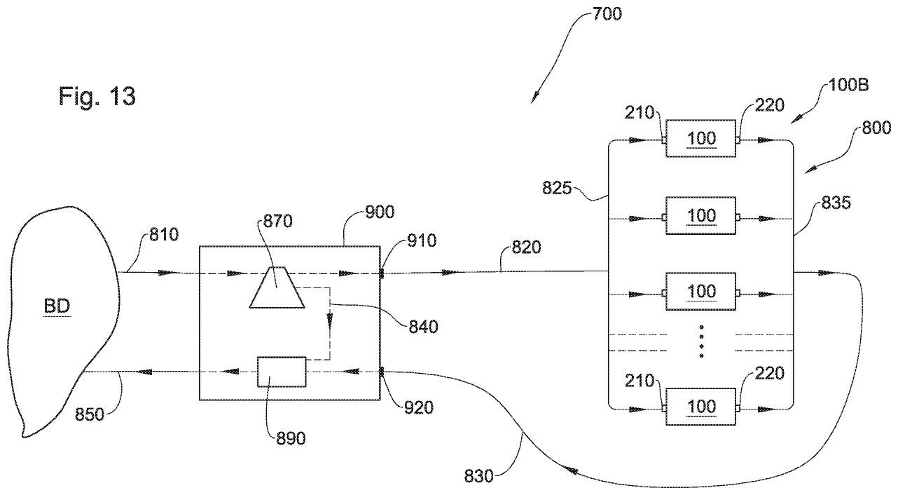

For example, in some other such examples, and referring to FIG. 13, the devices 100 in such a plurality (for example in a battery 100B of such devices) can be connected with respect to one another in parallel, such that the first conduit 810 is concurrently coupled to the fluid inlet port 210 of all the devices, for example via a first manifold 825. Similarly, the fluid outlet port 220 of all the devices 100 are concurrently coupled to the third conduit 830, and thus to the blood mixing reservoir 890, for example via a second manifold 835. Such an arrangement can allow for a larger volume flow of blood to be treated concurrently.

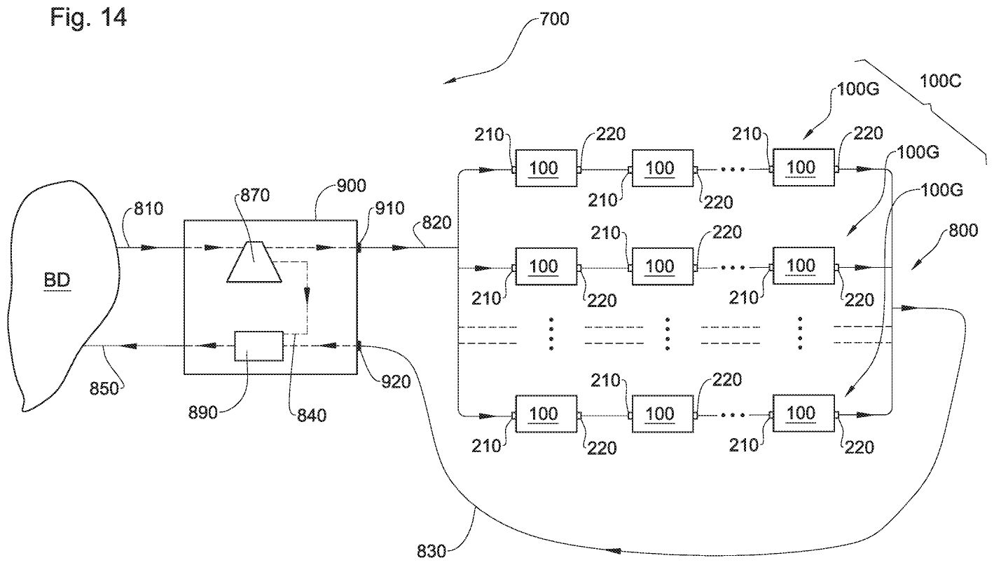

For example, in yet some other examples, and referring to FIG. 14 the devices 100 in such a plurality (for example in a battery 100C of such devices) can be interconnected both in parallel and in series, i.e., the devices are divided into groups 100G, the devices 100 in each group 100G are coupled to one another in series, and the groups 100G are coupled together in parallel. Each group 100G can include one, two three or more than three devices coupled in series, such that such that the fluid outlet port 220 of each upstream device is coupled to the fluid inlet port 210 of the next device 100 (directly or via tubing).

In at least this example, the active material 300 comprises at least one of a conjugate/s, a plurality of conjugates or at least one composition comprising the conjugates or plurality of conjugates. More specifically, the conjugate comprising a particle bonded to at least one linker comprising a chain of n carbon atoms covalently bonded to m carbonyl groups, and at least one trapping agent A covalently bonded to the m^{th} carbonyl group,

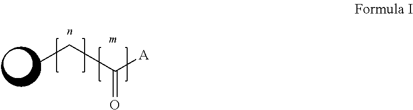

wherein, n is an integer within the range of 5 to 15, and m is an integer within the range of 5 to 10, wherein trapping agent A is characterized by having the ability to capture or bind amine. In some optional embodiments, the amine is at least one of methylamine, dimethylamine or trimethylamine.

It should be understood that the body of a subject in need thereof, as also referred to herein in FIGS. 10 to 14 as BD, refers to any mammalian subject in need of such treatment. In some embodiments, this subject (that may also in some embodiments referred to herein as a patient) is a subject having, or displaying elevated blood ammonia levels, and/or a subject suffering from any of the disorders and/r conditions associated with elevated blood ammonia levels, for example, hyperammonemia, and any of the conditions and disorders disclosed by the present disclosure in connection with other aspects.

In some embodiments, the device of the present disclosure may be used for depleting at least one amine from at least one liquid substance.

In yet some further specific embodiments, the amine depleted by the device of the present disclosure is ammonia. In some further embodiments, the liquid substance is a mammalian body fluid. Accordingly, the device of the present disclosure is used for depleting ammonia from mammalian body fluid/s.

In some embodiments, the conjugate comprised within the device are any of the conjugates defined by the present disclosure.

In some specific embodiments, the conjugate of the device of the invention is having the structural formula V. More specifically, the conjugate comprising a particle covalently bonded to at least one linker comprising a chain of 5, 6, 7, 8, 9, 10, 11, 12, 13, 14 15, or more carbon atoms, specifically, 15 carbon atoms covalently bonded to m carbonyl groups, and a sulfonic acid covalently bonded to the m^{th} carbonyl group.

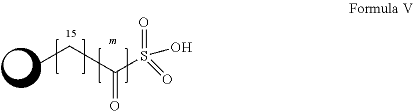

wherein m is an integer between 5 to 10. In some embodiments, the liner comprises a chain of 15 carbon atoms covalently bonded to 5 to 10 carbonyl groups, specifically, 5, 6, 7, 8, 9, 10 or more carbonyl groups.

In yet some further embodiment, the devices of the present disclosure are configured for housing a volume of at least about 50 ml to about 500 ml of the conjugate disclosed herein, or any plurality of conjugates or compositions thereof, specifically, about 50, 55, 60, 65, 70, 80, 85, 90, 95, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500 ml. In yet some further embodiments, the devices of the present disclosure are configured for housing a volume of at least about 250-350, specifically, 270-300 ml of the conjugates disclosed herein.

As will become clearer herein, the device is configured for depleting at least one ammonia from mammalian body fluids (for example human plasma and/or human whole blood and/or other mammalian plasma and/or other mammalian whole blood), as one example of treating mammalian body fluids. More specifically, the device disclosed herein is configured for depleting, reducing, partitioning ammonia from a body fluid.

The term “partitioning”, with respect to a target compound, specifically, at least one amine, and more specifically, ammonia, refers to the separation of the ammonia from the remainder of the liquid substance, specifically, blood fluid to provide a body fluid or any other liquid substance devoid of the ammonia. The term “partitioning” encompasses therefore depletion and removal of the at least one amine, and more specifically, ammonia from the liquid substance, specifically, body fluid. The term “depleting” or “depletion” as used herein is defined as the removal of the ammonia from the liquid substance, specifically, body fluid, to such extent so as to obtain an ammonia-depleted, or reduced liquid substance, specifically, body fluid. More specifically, the term “removal” or “depletion”, as used herein, either by partitioning or trapping of the at least one amine, specifically, ammonia, means the restriction, reduction, decrease or diminishing of the amount of the at least one amine, specifically, ammonia in the liquid substrate or body fluid by at least about 1%-100%, about 5%-95%, about 10%-90%, about 15%-85%, about 20%-80%, about 25%-75%, about 30%-70%, about 35%-65%, about 40%-60% or about 45%-55%. Said restriction, retardation, reduction, decrease or diminishing of the amount of at least one amine, specifically, ammonia in a liquid substance, specifically, body fluid, also be by at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or about 100%.

In yet some further embodiments, the devices of the present disclosure are adapted for depleting, removing and reducing t least one amine, specifically, ammonia from any volume of at least one liquid substance. More specifically, in some embodiments, the disclosed devices are particularly adapted for depleting ammonia from at least about 100 ml of body fluid, to at least about 10 liters of body fluid, specifically, 100, 200, 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000 ml, and mole, specifically, 5.5, 6, 6.5, 7, 7.5, 8. 8.5, 9, 9.5, 10 liter of body fluid.

A further aspect provided by the present disclosure relates to a battery for use in depleting ammonia from mammalian body fluid/s, comprising a plurality of devices defined by the present disclosure.

A further aspect relates to an extracorporeal apparatus comprising at least one conjugate, or at least one device comprising the conjugate. In some embodiments, the extracorporeal apparatus may be connected to such at least one device or battery of devices. In more specific embodiments, the conjugate of the extracorporeal apparatus of the present disclosure may comprise a particle bonded to at least one linker comprising a chain of n carbon atoms covalently bonded to m carbonyl groups, and at least one trapping agent A covalently bonded to the m^{th} carbonyl group,

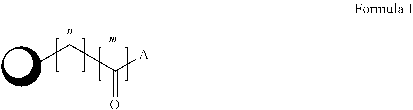

wherein, n is an integer within the range of 5 to 15, and m is an integer within the range of 5 to 10. More specifically, the trapping agent A is characterized by having the ability to capture or bind amine, optionally, the amine is at least one of methylamine, dimethylamine or trimethylamine. In yet some further embodiments, the device comprised within or connected to the extracorporeal apparatus may comprise:
a housing having at least one fluid inlet port, and at least one fluid outlet port;
the housing including at least one chamber, said at least one chamber defining a control volume in fluid communication with the at least one fluid inlet port and the at least one fluid outlet port. The control volume accommodating the at least one of a conjugate, a plurality of conjugates or at least one composition comprising the conjugates or plurality of conjugates.

In some embodiments, the conjugate/s, the plurality of conjugates or composition, the device and the battery used for the extracorporeal apparatus is as define by the present disclosure.

In some embodiments, the extracorporeal apparatus of the present disclosure is applicable for use in depleting ammonia from mammalian body fluid/s.

In yet some further embodiments, the extracorporeal apparatus of the present disclosure is adapted for depleting, removing and reducing t least one amine, specifically, ammonia from any volume of at least one liquid substance. More specifically, in some embodiments, the disclosed extracorporeal apparatus is particularly adapted for depleting ammonia from at least about 100 ml of body fluid, to at least about 10 liters of body fluid, specifically, between about 2 liters to 3 liters of a body fluid.

In a further aspect, the present invention provides a conjugate having the structural formula I, the conjugate comprising a particle bonded to at least one linker comprising a chain of n carbon atoms covalently bonded to m carbonyl groups, and at least one trapping agent A covalently bonded to the m^{th} carbonyl group.

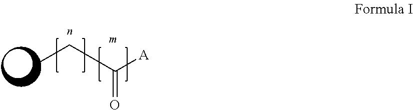

wherein, n is an integer within the range of 5 to 15, and m is an integer within the range of 5 to 10. In some embodiments, the trapping agent A is characterized by having the ability to capture or bind amine. As indicted above, in some embodiments the linker comprises 5 to 15 carbons. In some embodiments, since the length of one carbon atom is about 1.5 angstroms, thus, in some embodiments, the length of the linker may range between 7.5 or less to 22.5 or more, angstroms. In yet some further embodiments, the that in some embodiments, the linker further comprises between 5 to 10 carbonyls. As each carbonyl may be in the length of about 1.3 angstroms, the length may range between about 6.5 or less to about 13 angstroms or more.

In other words, the present disclosure provides a conjugate comprising three moieties:
particle;
a linker; and
trapping agent.

The three moieties are linked to each other in a way that the linker connects the particle and the trapping agent.

The linker of the invention generally includes 2 groups wherein the first group comprising a straight chain alkane comprising n carbon atoms and a group of m covalently bonded carbonyl groups.

In some embodiments, the straight chain alkane of the linker of the conjugate of the present disclosure is saturated or unsaturated.

In some embodiments, the straight chain alkane group may be saturated, where in other embodiments the group may be unsaturated.

In embodiments where the straight chain alkane group is unsaturated, the chain may contain between 1 to 3 double bonds.

The trapping agent moiety, designated herein as A, may include any agent having the ability to “capture” or bind amine.

In the context of the herein disclosure, the term amine relates to any compound or functional group containing at least one basic nitrogen atom with at least one lone pair of electrons. Amines according to the present disclosure may include any primary, secondary, and tertiary amines having a molecular weight (MW) of between at least 17 to at most 70 Daltons.

In some embodiments, the amine is an alkylamine, dialkylamine or trialkyl amine, wherein the MW of such amines is between 17 to 70 Daltons.

In some other embodiments, the amine is selected from methylamine, dimethylamine or trimethylamine.

In a specific embodiment, the amine is ammonia.

The linker of the invention is covalently linked to the trapping agent in a way that the m^{th} carbonyl is linked via straight linkage

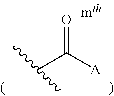

or through another short alkane chain

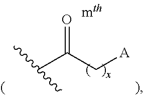

wherein X is an integer within the range of 1 to 3.

In some embodiments, the trapping agent may be any ion exchange material. More specifically, since ammonia is cationic in physiologic pH, it may bind a cation exchanger.

Other alternatives encompassed by the invention include, NHS and epoxy.

In some embodiments, the trapping agent is an acid.

In some embodiments the acid is a strong acid, capable of capturing an amine.

In some embodiments, the amine is as defined hereinabove.

In some other embodiments, the amine is ammonia.

In the context of the disclosure provided herein, the term “strong acid” is any acid having a pKa value which is lower than 1.

At times, the pKa is lower than 0, at times lower than (−1), at times lower than (−2), at times lower than (−3), at times lower than (−4), at times lower than (−5), at times lower than (−6), at times lower than (−7), at times lower than (−8), and at times lower than (−9).

In some specific embodiments, the acid is selected from the group consisting of chloric acid, hydrobromic acid, hydrochloric acid, hydroiodic acid, nitric acid, perchloric acid, sulfuric acid, hydroiodic acid, analogs thereof and derivates thereof.

In a specific embodiment, the acid is sulfuric acid or derivates thereof.

Derivates of sulfonic acid may be any molecule having the general formula:

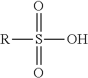

wherein R is an organic alkyl or aryl group.

In embodiments, the sulfonic acid derivate is selected from taurine, PFOS, p-Toluenesulfonic acid and coenzyme M.

In some embodiments, R is —H.

In most general terms, the length of the straight chain alkanes determines the specificity of the conjugate. A linker which is too long would capture unspecific/unwanted molecules such as proteins and amino acids (since they comprise an amino group). Short linkers would decrease the ability of the conjugate to capture ammonia and ammonium cations.

Thus, in embodiments, the length of the straight chain alkanes (n) is between 5-20 carbon atoms, specifically, 5-19, 5-18, 5-17, 5-16, 5-15, 5-14, 5-13, 5-12, 5-11, 5-10, 6-20, 7-20, 8-20, 9-20, 10-20, 1-20, 12-20, 13-20, 14-20, 15-20. In yet some further embodiments, the length of the straight chain alkanes (n) is at times, between 10-15, at times between 12-15, at times between 13-15, at time between 14-15. In some specific embodiments, the length of the straight chain alkanes (n) is 5 or less, 6, 7, 8, 9, 10, 11, 12, 13, 14, 15, 16, 17, 18, 19, 20, or more. In a specific and non-limiting embodiment, n is 15.

Without wishing to be bound by theory or mechanism, the carbonyls moiety provides the appropriate bulkiness which prevents the attachment of amino acids and peptides to the acid moiety of the conjugate. The inventors of the present disclosure surprisingly discovered that a group of between 5-10 carbonyls bonded together, provides the adequate bulkiness which prevents bigger molecules from being captured by the acidic moiety of the conjugate.

Thus, in embodiments, the conjugates of the present disclosure (as referred to herein in all the disclosed Formula, and all disclosed aspects) comprise between about 5 to 10 carbonyl groups (m), specifically, 5-10, 5-9, 5-8, 5-7, 5-6, 6-10, 6-9, 6-8, 6-7, 7-10, 7-9, 7-8, 5-10, 8-9. More specifically, 5, 6, 7, 8, 9, or 10 carbonyl groups. In a specific and non-limiting embodiment, m is 5, according to such embodiments, the sulfonic acid covalently bonded to the m^{th} carbonyl group, is meant that the sulfonic acid covalently bonded to the 5^{th} carbonyl group. In a specific and non-limiting embodiment, m is 6, according to such embodiments, the sulfonic acid covalently bonded to the m^{th} carbonyl group, is meant that the sulfonic acid covalently bonded to the 6^{th} carbonyl group. In a specific and non-limiting embodiment, m is 7, according to such embodiments, the sulfonic acid covalently bonded to the m^{th} carbonyl group, is meant that the sulfonic acid covalently bonded to the 7^{th} carbonyl group. In a specific and non-limiting embodiment, m is 8, according to such embodiments, the sulfonic acid covalently bonded to the m^{th} carbonyl group, is meant that the sulfonic acid covalently bonded to the 8^{th} carbonyl group. In a specific and non-limiting embodiment, m is 9, according to such embodiments, the sulfonic acid covalently bonded to the m^{th} carbonyl group, is meant that the sulfonic acid covalently bonded to the 9^{th} carbonyl group. In a specific and non-limiting embodiment, m is 10, according to such embodiments, the sulfonic acid covalently bonded to the m^{th} carbonyl group, is meant that the sulfonic acid covalently bonded to the 10^{th} carbonyl group.

It should be understood that in certain embodiments, the conjugate of the present disclosure may comprise any combination of any number (m) of carbonyl group with any number of straight chain alkanes (n).

In other embodiments of the invention there is provided a conjugate comprising a particle bonded to at least one linker comprising a chain of 15 carbon atoms covalently bonded to between 5 to 10 carbonyl groups (m), and an acid A covalently bonded to the m^{th} carbonyl group, the conjugate having the structural formula II:

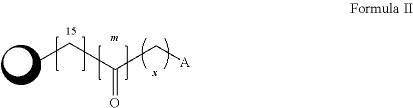

wherein x is between 0 to 3.

When x is 0, the m^{th} carbonyl is directly connected to the acid.

In some further embodiments, the invention provides a conjugate comprising a particle bonded to at least one linker comprising a chain of 15 carbon atoms covalently bonded to between 5 to 10 carbonyl groups (m), and a sulfonic acid covalently bonded to the m^{th} carbonyl group, the conjugate having the structural formula III:

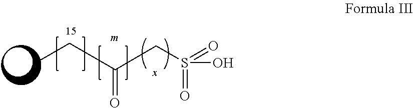

wherein x is between 0 to 3.

In some embodiments, x is between 0 to 2, at times between 0 to 1. At times x is 1, and at times x is 0.

When x is 0, the m^{th} carbonyl is directly connected to the acid.

In some embodiments, the conjugate of the present disclosure is configured to act as an amine trap, exhibiting a neutralization reaction of amines. In more specific embodiments, the amine is ammonium.

Still further, in some embodiments, the linker moiety of the conjugate of the present disclosure is connected to the particle via any suitable functional group allowing connecting the particle to the straight chain alkane.

Without being bound by theory or reaction mechanism, conjugates of the invention act as an amine trap exhibiting a neutralization reaction of one amine as defined above for each acid trapping agent, in the following manner

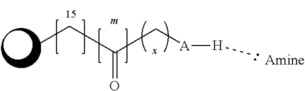

wherein —H is the free hydrogen atom of the acid and the (:) are the free electrons of the amine.

In embodiments where the acid is sulfonic acid and the amine is ammonia, a proton (Fr) is donated to the two free electrons of ammonia as follows:

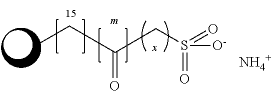

generating an ionic bond between the conjugate acid and conjugate base.

Linker moiety of the herein disclosure may be connected to the particle via any group known in the art, which allows connecting the particle to the straight chain alkane.

In one possible embodiment, the particle and the linker and covalently connected.

In another optional embodiments, the linkage is a covalent linkage via amino group as presented in Formula IV:

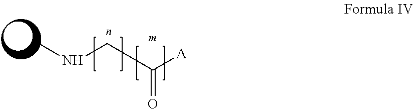

In yet some other embodiments, the invention contemplates a conjugate having the structural formula V, wherein n is 15, m is an integer between 5 to 10, x is 0 and A is a sulfonic acid:

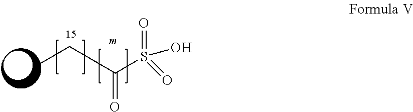

The preset invention provides at least one conjugate. A conjugate as used herein refers to a compound constructed from several elements (components), including at least one particle, at least one linker and at least one trapping agent, specifically a trapping agent for ammonia, that may be in some embodiments a sulfonic acid or any derivative thereof or an analog thereof which are all associated thereto. It should be noted that while the application refers to a “at least one particle”, any solid support being applicable for the claimed plurality of conjugate is encompassed herein.

Any one of the conjugates of the presently disclosed subject-matter or any compositions thereof, may also be referred to as a composition of matter. In most general terms, a “composition of matter” similarly to a “conjugate”, both used interchangeably, refers to the association of the at least one particle, the at least one linker and the at least one trapping agent, derivative thereof or analog thereof, that as detailed below produces properties which may be attributed to the composition of matter (or conjugate) as a whole and not to any one of conjugate's components in their separate state.

In some embodiments, any one of the conjugates of the presently disclosed subject-matter encompasses an association of the at least one particle with the at least one chemically reactive moiety being a linker and the association of the at least one linker with the at least one trapping agent, specifically, sulfonic acid and derivative thereof or an analog thereof such that the linker is positioned between the particle and the trapping agent, derivative thereof or an analog thereof and hence being associated at one end (at one arm) to the particle and at another end (at a second different arm) to the trapping agent, derivative thereof or an analog thereof.

In some embodiments, a trapping agent, specifically, sulfonic acid and derivative thereof, binds specifically and selectively to a particular target, in this case at least one amine, for example, ammonia and enables effective trapping, immobilization, partitioning and removal of the ammonia from the liquid substance, specifically, body fluid.

As used herein, the term “association” or any linguistic variation thereof refers to the chemical or physical force which holds two entities together (e.g., the particle and the linker). Such force may be any type of chemical or physical bonding interaction known to a person skilled in the art.

Non-limiting examples of such association interactions are covalent bonding, ionic bonding, coordination bonding, complexation, hydrogen bonding, van der Waals bonding, hydrophobicity-hydrophilicity interactions, etc. Thus, the association/conjugation of the linker with the at least one particle and of the linker with the trapping agent may be via any chemical bonding, including covalent bonding, electrostatic interaction, acid base interaction, van der Waals interaction, etc. As appreciated, the association of the particle and the linker and the association of the linker with the trapping agent, a derivative thereof or an analog thereof may be the same or may be different as further detailed below.

Particles of the invention may include any polymeric particle which is able to bind the linker of the invention.

In some embodiments, the particle is a resin bead.

The term particles as used herein refers to a portion of matter having a surface that can be attached to chemical or biological compounds, small or large molecules that may be attached through either covalent or non-covalent bonds. The particle may comprise a porous material. The particles may be “spherical” (refers generally to a substantially (nearly) round-ball geometry) or “non-spherical”, for example, (“elongated” in shape and has a defined long and short axis). Non-liming examples of particles include beads such as at least one of polysaccharide bead, glass beads, cotton bead, plastic bead, nylon bead, latex bead, magnetic bead, paramagnetic bead, super paramagnetic bead, starch bead and the like, silicon bead, PTFE bead, polystyrene bead, gallium arsenide bead, gold bead, or silver bead. In some embodiments, the particle is a bead comprising agarose beads, optionally at different degree of crosslinking at different % of material (agarose).

As such, agarose beads encompass beads comprising agarose at varying degree of crosslinking, for example beads denoted as Sepharose beads. In some embodiments, the bead comprises agarose beads. In some embodiments, the bead comprises Sepharose beads. In some embodiments, the plurality of conjugates comprises a combination of particles comprising agarose beads and Sepharose beads. In accordance with the present disclosure, it should be noted that particles being either agarose beads and Sepharose beads are considered as two different conjugates having different particles.

The particle and specifically the bead as described herein may be associated to a chemically reactive moiety, denoted herein as a linker. The linker as used herein may be any chemical entity that is composed of any assembly of atoms, including oligomeric and polymeric chains of any length, which according to some embodiments, is capable of binding on one end to the particle and on the other end the at least one trapping agent, a derivative thereof or an analog thereof.

In some embodiments, the bead may be associated to the linker via a spacer or coating present on the bead. As such, the bead is initially activated (“activated beads”) by association to a spacer/coating and then reacted with a linker. It should be noted that at times, no linker may be further required in cases the spacer/coating binds directly to the at least one trapping agent. At times, the bead does not have a functional group capable of binding to the linker, and a spacer or coating may be used.

The activated beads are obtained by pre-coating the beads with a suitable material having an active moiety enabling the binding to the beads and to the linker and/or the trapping agent. In other words, the beads are pre-coated to include reactive groups enabling the covalent binding to either the linker or the trapping agent.

In some embodiments the beads may be activated for example by pre-coating with any coating material. Non-limiting examples of such material include for example, amino acid, protein, epoxy, tosyl, carboxylic acid, carboxylated polyvinyl alcohol. when referring to “pre-coating” it should be understood as a preliminary step which results in coating of the beads with an active material that in turn enables covalent binding of the beads with the sulfonic acid (i.e., directly) or via at least one linker. In some embodiments, the beads are pre-coated with an amino acid, peptide or any derivative thereof. Pre-coated magnetic beads may comprise for example as active groups, a primary amine (—NH2), carboxyl (—COOH), sulfhydryl (—SH) or carbonyl (—CHO). In some embodiments, the beads are pre-coated to include a moiety that may react with primary or secondary amino groups. In some other embodiments, the magnetic beads are coated with polylysine.

As used herein the term “linker” encompasses any spacer or pre-coating present on the beads.

In some embodiments, the linker comprises or is a chain of atoms, for example a linear chain. In some embodiments, the linker comprises at least 1 atom, at least 4 atoms, at times 5 atoms, at times 10 atoms, at times 20 atoms, at times 30 atoms, at times 40 atoms. In some embodiments the linker is or comprises a linear chain of 1 to 40 atoms. In some embodiments the linker is or comprises a linear chain of 1 atom. In some embodiments the linker is a linear chain comprising 5 atoms. In some embodiments the linker is a linear chain comprising 15 atoms.

In some embodiments, the particle is a resin bead. In some embodiments, the particle may be an agarose bead, and therefore, the resin bead is in some embodiments the agarose resin may comprise between about 2% to 10% agarose. Still further, in some embodiments the resin bead may comprise between 3% to 9% agarose, still further in some embodiments, the resin bead may comprise between 4% to 8% agarose. In some embodiments, the resin bead comprising at least 4% of agarose.

In some other embodiments, the amount of agarose in the particle is at least 5%, and at times at least 6%.

In some embodiments, the conjugate of the resent disclosure comprises particles having an average particle size of between about 10 μm or less, to about 500 μm or more. Specifically, 10 μm, 20 μm, 30 μm, 40 μm, 50 μm, 60 μm, 70 μm, 80 μm, 90 μm, 100 μm, 110 μm, 120 μm, 130 μm, 140 μm, 150 μm, 160 μm, 170 μm, 180 μm, 190 μm, 200 μm, 250 μm, 300 μm, 350 μm, 400 μm, 450 μm, 500 μm or more. In some specific embodiments, the plurality of conjugates comprise particles having an average particle size of at least 70 μm or less, at times at least 80 μm, at times at least 90 μm, at times at least 100 μm, at times at least 110 μm, at times at least 120 μm, at times at least 130 μm, at times at least 140 μm, at times at least 150 μm. In some embodiments, the plurality of conjugates display an average particle size of between about 40 μm or less, to about 170 μm or more.

The term “average size” or “average diameter” or “mean size” refers to the arithmetic mean of measured diameters, wherein the diameters range ±25% of the mean. The mean size of the particles can be measured by any method known in the art. In certain embodiments, the size of the resin bead is between 40-170 μm, where the average size is between 80-100 μm. At times, the average size is 90 μm.

In some embodiments, the plurality of conjugates according to the present disclosure may be particularly applicable for use in depleting at least one amine from at least one liquid substance.

In yet some further embodiments, the trapping agent A of the conjugate of the plurality of conjugates in accordance with the present disclosure is specifically configured for capturing at least one amine. In some embodiments, the amine is ammonia. In yet some further embodiments, the liquid substance is a mammalian body fluid. Thus, in some embodiments, the plurality of conjugates provided by the present disclosure is specifically applicable for use in depleting ammonia from mammalian body fluid/s.

In some embodiments, the plurality of conjugates according to the present disclosure may be particularly applicable for use in depleting at least one amine from at least one liquid substance.

A further aspect of the present disclosure relates to a plurality of conjugates or any composition comprising the plurality of conjugates. In more specific embodiments, each conjugate comprises a particle, at least one linker and at least one trapping agent A, or any derivative or analog thereof. More specifically, the conjugate of the present disclosure comprises a particle bonded to at least one linker comprising a chain of n carbon atoms covalently bonded to m carbonyl groups, and at least one trapping agent A covalently bonded to the m^{th} carbonyl group;

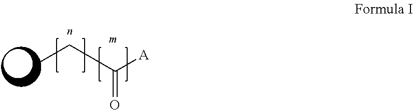

wherein, n is an integer within the range of 5 to 15, and m is an integer within the range of 5 to 10. In some embodiments, the trapping agent A is characterized by having the ability to capture or bind amine Optionally, the amine is at least one of methylamine, dimethylamine or trimethylamine.

In some embodiments, the conjugate of the plurality of conjugates or in any composition disclosed by the present invention, is as defined by the present disclosure.

In some embodiments, the plurality of conjugates according to the present disclosure may be particularly applicable for use in depleting at least one amine from at least one liquid substance.

In yet some further embodiments, the trapping agent A of the conjugate of the plurality of conjugates in accordance with the present disclosure is specifically configured for capturing at least one amine. In some embodiments, the amine is ammonia. In yet some further embodiments, the liquid substance is a mammalian body fluid. Thus, in some embodiments, the plurality of conjugates provided by the present disclosure is specifically applicable for use in depleting ammonia from mammalian body fluid/s.

A further aspect of the resent disclosure relates to a method for depleting at least one amine from a liquid substance. More specifically, the method comprising the steps of: In a first step (i), subjecting the liquid substance to affinity-depletion procedure specific for the at least one amine. The next step (ii) involves recovering the at least one amine-depleted liquid obtained in step (i). In some embodiments, the affinity-depletion procedure comprises contacting the liquid substance with an effective amount of at least one conjugate, a plurality of conjugates or with a composition comprising the conjugate or plurality of conjugates, or applying the liquid substance on a device, battery, or extracorporeal apparatus comprising the conjugates of the present disclosure. In more specific embodiments, each conjugate comprises a particle bonded to at least one linker comprising a chain of n carbon atoms covalently bonded to m carbonyl groups, and at least one trapping agent A covalently bonded to the m^{th} carbonyl group,

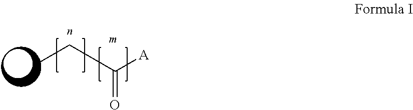

wherein, n is an integer within the range of 5 to 15, and m is an integer within the range of 5 to 10, wherein trapping agent A is characterized by having the ability to capture or bind the amine. Optionally, the amine is at least one of methylamine, dimethylamine or trimethylamine.

It should be understood that in certain embodiments, the conjugate of the present disclosure may comprise any combination of any number (m) of carbonyl group with any number of straight chain alkanes (n). Thus, in some embodiments, the plurality of conjugates as discussed herein may comprise any mixture or combination of any conjugate that combines 5 to 15 straight chain alkanes (n), with 5 to 10 carbonyl group.

In some embodiments, the liquid substance used in the methods of the present disclosure is a mammalian body fluid/s or any products thereof.

In yet some further embodiments, the methods of the invention are used for depleting at least one amine from any liquid substance. In some embodiments, the amine is ammonia. Thus, in some embodiments, the methods of the present disclosure are for use in depleting ammonia from a mammalian body fluid. As indicated above, the methods of the present disclosure are adapted for depleting and reducing ammonia from at least about 0.5 liter to about 10 liters of body fluids, specifically, between about 2-3 liters of body fluid.

In should be noted that in some embodiments, any of the conjugate/s, plurality of conjugates or composition, device and/or battery used by the methods discussed herein are as defined by the invention.

As indicated above, the methods of the invention involve extracorporeal procedures. In some embodiments, the extracorporeal apparatus is a cardiopulmonary bypass machine (CPB), and wherein the extracorporeal apparatus is a plasmapheresis machine.

The term “extracorporeal” refers to a medical procedure which is performed outside the body. For example, such extracorporeal procedure may relate to a circulatory procedure i.e. a procedure in which blood is taken from a patient's circulation to have a process applied to it before it is returned to the circulation. All of the apparatus carrying the blood outside the body is termed the extracorporeal circuit. Such circulatory procedures include for example but are not limited to Apheresis, Autotransfusion, Hemodialysis, Hemofiltration, Plasmapheresis, Extracorporeal carbon dioxide removal, Extracorporeal cardiopulmonary resuscitation, Extracorporeal membrane oxygenation (ECMO) and Cardiopulmonary bypass during open heart surgery.

Cardiopulmonary bypass (CPB) is a technique that temporarily takes over the function of the heart and lungs during surgery, maintaining the circulation of blood and the oxygen content of the patient's body. The CPB pump itself is often referred to as a heart-lung machine or “the pump”. Cardiopulmonary bypass pumps are operated by perfusionists. CPB is a form of extracorporeal circulation. Extracorporeal membrane oxygenation is generally used for longer-term treatment.

An apheresis machine is a device which receives blood removed from a body of a subject in need thereof, and separates it into its various components: plasma, platelets, white blood cells and red blood cells.

A further aspect of the invention relates to a method for depleting at least one amine from body fluid/s of a subject in need thereof. More specifically, the method may comprise contacting the body fluid with an effective amount of conjugates, a plurality of conjugates or a composition thereof, or within a device or battery comprising the conjugate, or alternatively, with an extracorporeal apparatus that comprises the conjugate o device described herein or connected to the conjugate or device disclosed herein. It should be noted that each conjugate comprises a particle bonded to at least one linker comprising a chain of n carbon atoms covalently bonded to m carbonyl groups, and at least one trapping agent A covalently bonded to the m^{th} carbonyl group,

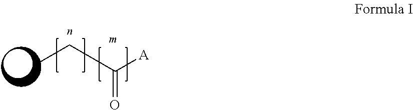

wherein, n is an integer within the range of 5 to 15, and m is an integer within the range of 5 to 10, wherein trapping agent A is characterized by having the ability to capture or bind amine Optionally, the amine is at least one of methylamine, dimethylamine or trimethylamine. The next step involves recovering the amine-free body fluid, and optionally, re introducing the body fluid to the subject in need.

In yet some further specific and non-limiting embodiments, the method may comprise the use of an extracorporeal procedure. More specifically, such method may comprise the steps of:

First in step (i), transferring body fluids of the subject into an extracorporeal apparatus. The next step (ii), involves subjecting the body fluid to affinity depletion procedure specific for at least one amine, wherein said depletion is performed before, during or after blood is being transferred into and out-off said apparatus, thereby obtaining an extracorporeal body fluid of said subject depleted in at least one amine.

The next step (iii) involves reintroducing or returning said body fluid obtained in step (ii) to the subject. As indicated above, the affinity-depletion procedure comprises contacting the body fluid of the subject with an effective amount of conjugates, a plurality of conjugates or a composition thereof comprised within said extracorporeal apparatus, or within a device or battery connected to the extracorporeal apparatus. Each conjugate comprises a particle bonded to at least one linker comprising a chain of n carbon atoms covalently bonded to m carbonyl groups, and at least one trapping agent A covalently bonded to the m^{th} carbonyl group,

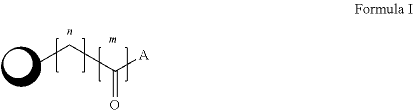

wherein, n is an integer within the range of 5 to 15, and m is an integer within the range of 5 to 10, wherein trapping agent A is characterized by having the ability to capture or bind amine Optionally, said amine is at least one of methylamine, dimethylamine or trimethylamine.

In some embodiment, the conjugate, plurality of conjugates or composition, device, battery and apparatus used by the methods of the invention are any of those disclosed by the present disclosure.

In some embodiments, the conjugate used by the methods of the present disclosure is having the structural formula V, the conjugate comprising a particle covalently bonded to at least one linker comprising a chain of 15 carbon atoms covalently bonded to m carbonyl groups, and a sulfonic acid covalently bonded to the m^{th} carbonyl group

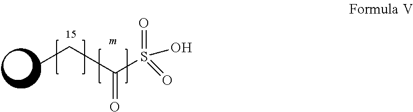

wherein m is an integer between 5 to 10.

In some embodiments, the methods of the presently disclosed subject-matter may be applicable for depleting ammonia from any liquid material or substance, specifically, from body fluids. Body fluids, bodily fluids, or biofluids, as used herein, are liquids within the mammalian body, specifically, the human body. This term may refer is some embodiments to any body-fluid, including blood, blood plasma, saliva, vaginal fluids, semen, urine, mucosa, and the like, but in the context of the present disclosure refer to blood and blood plasma as discussed below. The average total body water is about 60% (60-67%) of the total body weight; it is usually slightly lower in women (52-55%). The exact percentage of fluid relative to body weight is inversely proportional to the percentage of body fat. The total body of water is divided into fluid compartments, between the intracellular fluid (ICF) compartment (also called space, or volume) and the extracellular fluid (ECF) compartment (space, volume) in a two-to-one ratio: 28 (28-32) liters are inside cells and 14 (14-15) liters are outside cells. The ECF compartment is divided into the interstitial fluid volume, the fluid outside both the cells and the blood vessels, and the intravascular volume (also called the vascular volume and blood plasma volume), the fluid inside the blood vessels, in a three-to-one ratio: the interstitial fluid volume is about 12 liters, the vascular volume is about 4 liters.

In some embodiments, the body fluids referred to herein is blood plasma. Blood plasma is the liquid component of blood that is freed from blood cells, but holds proteins and other constituents of whole blood in suspension. It makes up about 55% of the body's total blood volume. It is the intravascular part of extracellular fluid as discussed above. Plasma is mostly composed of water (up to 95% by volume), and contains important dissolved proteins (6-8%) (e.g., serum albumins, globulins, and fibrinogen), glucose, clotting factors, electrolytes (Na⁺, Ca²⁺, Mg²⁺, HCO₃⁻, etc.), hormones, carbon dioxide, and oxygen. Blood plasma has a density of approximately 1025 kg/m³, or 1.025 g/ml. Still further, in some embodiments, the body fluid useful in the present disclosure may be a blood serum, that is blood plasma as discussed herein, without clotting factors.

In some embodiments, the methods of the presently disclosed subject-matter may be applicable for depleting ammonia from body fluid that may be at least one of whole blood, plasma or blood-derived product.

In some specific embodiments, such blood-derived product may be at least one of whole blood, plasma, fresh frozen plasma (FFP), platelet rich plasma (PRP) and cryoprecipitate. It should be understood that in some embodiments, the method of the presently disclosed subject matter may be performed ex vivo or in vitro. More specifically, in body fluids that are no longer part of the human body.

The present disclosure provides conjugates, devices and methods for depleting ammonia from body fluid, thereby obtaining ammonia-reduced, ammonia-depleted body fluid. An “ammonia-depleted, or reduced, body fluid” or “ammonia-free body fluid” as used herein is meant that the products of the presently disclosed subject-matter (that according to some embodiments, have been prepared by treating body fluid such as blood, plasma or blood products with the ammonia binding agent, specifically, the device and conjugates disclosed by the present invention), display a reduced, decreased, attenuated, amount of ammonia in about 50% to 100%, as compared to untreated blood or blood product. More specifically, at least about 50%, 55%, 60%, 65%, 70%, 75%, 80%, 85%, 90%, 95%, 96%, 97%, 98%, 99% or 100%, of ammonia initially, or normally present in body fluid, specifically, blood or blood products before depletion using the methods, devices and conjugates disclosed herein, is removed from the products of the presently disclosed subject-matter, specifically when compared to untreated blood or blood products. In other words, the product of the presently disclosed subject-matter may comprise ammonia in an amount of about 0.01% to about 50% of the amount of the ammonia in other products or untreated blood or blood products, specifically, blood, plasma, o blood product that were not subjected to the conjugates, devices and methods of the present disclosure. Specifically, about 0.01% or less, 0.05%, 0.1%, 0.5%, 1%, 2%, 3%, 4%, 5%, 6%, 7%, 8%, 9%, 10%, 15%, 20%, 25%, 30%, 35%, 40%, 45%, 50%, 60%, 70% or less of the amount of ammonia as compared to untreated blood or blood products.

As indicated above, the body fluid, blood, plasma or blood products treated by the methods of the presently disclosed subject-matter display reduced or decreased or depleted amount of ammonia. It should be appreciated that the terms “reduced” or “decreased” as referred to herein, relate to the decrease or reduction of the amount of at least one amine, specifically ammonia, by any one of about 1% to 99.9%, specifically, about 1% to about 5%, about 5% to 10%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 60% to 65%, about 65% to 70%, about 75% to 80%, about 80% to 85% about 85% to 90%, about 90% to 95%, about 95% to 99%, or about 99% to 99.9%, or even 100% as compared to body fluids such as blood, plasma or blood products that comprise ammonia, to blood or blood products that were not treated with the conjugates of the presently disclosed subject-matter, to blood of subjects suffering from conditions associated with elevated levels of ammonia, and in some embodiments, to normal blood or blood products or to commercially available blood products. In other words, these products display no ammonia, or at the most, minimal and reduced amount of ammonia, specifically, about 0.01% or less, 0.05%, 0.1%, 0.5%, 1%, 2%, 3%, 4%, 5%, 6%, 7%, 8%, 9%, 10%, 15%, 20%, 25%, 30%, 35%, 40%, 45%, 50% or less of the amount of ammonia as compared to the amount of ammonia of an untreated blood, plasma or blood product or any other body fluid. In some embodiments, the body fluid, blood, plasma or blood products treated by the methods, conjugates, compositions, as well as the device, battery, kits or systems provided by the presently disclosed subject-matter, that display reduced or no ammonia as defined above, may be used for any therapeutic applications disclosed by the presently disclosed subject-matter, as discussed herein after.

In yet some other specific embodiments, the method of the presently disclosed subject-matter may be used in vivo/ex vivo for depleting at least one ammonia from body fluid/s of and/or in a subject in need thereof.

A further aspect of the present disclosure relates to a method for the treatment, prevention, prophylaxis, amelioration, inhibition of disorders associated with elevated blood ammonia levels or pathologic condition associated therewith in a subject in need thereof by depleting ammonia from body fluid/s of a subject in need thereof.

More specifically, the therapeutic methods disclosed herein may comprise contacting the body fluid of the treated subject with an effective amount of conjugates, a plurality of conjugates or a composition thereof, or within a device or battery comprising the conjugate, or alternatively, with an extracorporeal apparatus that comprises the conjugate and/or device described herein or connected to the conjugate or device disclosed herein. It should be noted that each conjugate comprises a particle bonded to at least one linker comprising a chain of n carbon atoms covalently bonded to m carbonyl groups, and at least one trapping agent A covalently bonded to the m^{th} carbonyl group,

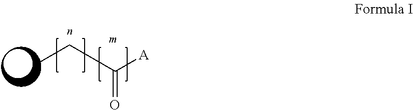

wherein, n is an integer within the range of 5 to 15, and m is an integer within the range of 5 to 10, wherein trapping agent A is characterized by having the ability to capture or bind amine Optionally, the amine is at least one of methylamine, dimethylamine or trimethylamine. The next step involves recovering the amine-free body fluid, and optionally, reintroducing the body fluid to the treated subject.

In yet some further specific and non-limiting embodiments, the methods may comprise the use of an extracorporeal procedure. More specifically, such method may comprise the steps of:

First in step (i), transferring body fluids of the subject into an extracorporeal apparatus. The next step (ii), involves subjecting the body fluid to affinity depletion procedure specific for at least one amine, wherein said depletion is performed before, during or after blood is being transferred into and out-off said apparatus, thereby obtaining an extracorporeal body fluid of the treated subject depleted in at least one amine.

The next step (iii) involves reintroducing or returning said body fluid obtained in step (ii) to the subject. As indicated above, the affinity-depletion procedure comprises contacting the body fluid of the subject with an effective amount of conjugates, a plurality of conjugates or a composition thereof comprised within said extracorporeal apparatus, or within a device or battery connected to the extracorporeal apparatus. Each conjugate comprises a particle bonded to at least one linker comprising a chain of n carbon atoms covalently bonded to m carbonyl groups, and at least one trapping agent A covalently bonded to the m^{th} carbonyl group,

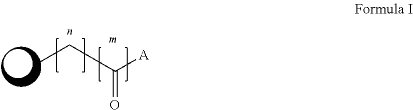

wherein, n is an integer within the range of 5 to 15, and m is an integer within the range of 5 to 10, wherein trapping agent A is characterized by having the ability to capture or bind amine Optionally, said amine is at least one of methylamine, dimethylamine or trimethylamine.

In some embodiment, the conjugate, plurality of conjugates or composition, device, battery and apparatus used by the therapeutic methods of the invention are any of those disclosed by the present disclosure.

In some embodiments, the conjugate used by the therapeutic methods of the present disclosure is having the structural formula V, the conjugate comprising a particle covalently bonded to at least one linker comprising a chain of 15 carbon atoms covalently bonded to m carbonyl groups, and a sulfonic acid covalently bonded to the m^{th} carbonyl group

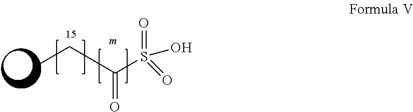

wherein m is an integer between 5 to 10.

In some embodiments, the methods, systems, devices, apparatus and conjugates of the present disclosure may be applicable for any disorders associated with elevated blood ammonia levels is a chronic or acute hepatic conditions, and/or chronic or acute pulmonary condition, cognitive decline and any other disorders associated with neuronal and/or neurological damage, and hyperammonemia and associated conditions.

In some specific embodiments, hepatic condition is hepatic encephalopathy and any related conditions. Hepatic encephalopathy is a decline in brain function that occurs as a result of severe liver disease. In this condition, your liver cannot adequately remove toxins from the blood of the subject. This causes a buildup of toxins in the bloodstream, which can lead to brain damage.

Hepatic encephalopathy can be acute (short-term) or chronic (long-term). In some cases, a person with hepatic encephalopathy may become unresponsive and slip into a coma.

Acute hepatic encephalopathy may also be a sign of terminal liver failure. Chronic hepatic encephalopathy may be permanent or recurrent.

Still further, hepatic encephalopathy is a syndrome usually observed in patients with cirrhosis. Hepatic encephalopathy is defined as a spectrum of neuropsychiatric abnormalities in patients with liver dysfunction, after exclusion of brain disease. Hepatic encephalopathy is characterized by personality changes, intellectual impairment, and a depressed level of consciousness. An important prerequisite for the syndrome is diversion of portal blood into the systemic circulation through portosystemic collateral vessels. Hepatic encephalopathy is also described in patients without cirrhosis with either spontaneous or surgically created portosystemic shunts. The development of hepatic encephalopathy is explained, to some extent, by the effect of neurotoxic substances, which occurs in the setting of cirrhosis and portal hypertension.

In some embodiments, the methods, systems, devices, apparatus and conjugates of the present disclosure may be applicable for any condition, symptom or disorder associated with hyperammonemia. Hyperammonemia, as used herein, is the pathological accumulation of ammonia in the blood, which can occur in many different clinical settings. Most commonly in adults, hyperammonemia occurs secondary to hepatic dysfunction; however, it is also known to be associated with other pathologies, surgeries, and medications. Although less common, hyperammonemia has been described as a rare, but consistent complication of solid organ transplantation. Lung transplantation is increasingly recognized as a unique risk factor for the development of this condition, which can pose grave health risks, including long-term neurological sequelae and even death. A wide array of etiologies has been attributed to this condition. A growing number of case studies and investigations suggest disseminated opportunistic infection with Ureaplasma or Mycoplasma species may drive this metabolic disturbance in lung transplant recipients. Regardless of the etiology, hyperammonemia presents a severe clinical problem with reported mortality rates as high as 75%. Surviving patients are often burdened with significant long-term neurological sequelae, such as cognitive impairment. It is therefore appreciated that the methods, systems, devices, apparatus and conjugates of the present disclosure may be applicable for the treatment and prevention of hyperammonemia, specifically following solid organ transplantations. In some embodiments, the disclosed methods, systems, devices, apparatus and conjugates of the present disclosure, are applicable for patients undergoing lung transplantation.

Still further, ammonia (NH3) is an ordinary metabolite in the human body, however, supraphysiologic levels in the systemic circulation can result in profound neurological damage and even death.

In children, it is classically associated with inborn-errors-of-metabolism concerning urea cycle enzymes and transporters, collectively called urea cycle disorders (UCDs). It is therefore appreciated that in some embodiments, the methods, systems, devices, apparatus and conjugates of the present disclosure may be applicable for treating and/or ameliorating UCDs.

Still further, in some embodiments, the methods, systems, devices, apparatus and conjugates of the present disclosure may be applicable for chronic kidney disease, hemorrhagic shock, and any hyperammonemia associated disorder well understood and reported in the literature.

Still further, it should be understood that in all cases of hyperammonemia, regardless of the etiology, the mechanism of ammonia's effect on the central nervous system is the same. Once in the systemic circulation, NH3 is able to cross the blood brain barrier through multiple mechanisms including gaseous diffusion, passive diffusion in its soluble form through membrane channels, and competitively through potassium channels. In the brain, NH3 is taken up by astrocytes and converted to glutamine through the action of glutamine synthetase (GS). This results in a series of adverse events. The markedly elevated Gln increases the osmotic pressure, causing a disruption of aquaporins, ultimately leading to the cerebral edema and hypertension characteristic of hyperammonemia. Concomitantly, astrocytes release various pro-inflammatory cytokines, such as tissue necrosis factor alpha (TNF-α), Astrocyte impairment and subsequent downregulation of their glutamate receptors can trigger excessive glutamatergic activity in adjacent synapses, leading to excitotoxicity that results in the encephalopathy and seizures commonly seen in hyperammonemia.

Similar physiologic changes of increased GAB Aergic tone occur in the Purkinje cells of the cerebellum, which is likely responsible for the ataxia and myoclonus also seen with this metabolic disturbance. Delay in recognition and proper management can lead to significant long-term morbidity, including refractory status epilepticus, motor and cognitive impairment, cerebral palsy, and death. Thus, in some embodiments, the methods, systems, devices, apparatus and conjugates of the present disclosure ae applicable for any of the disclosed neuronal disorders, and any related conditions. In adults, for example, such conditions may be further characterized with altered mental state, lethargy, disturbances in mood and personality, ataxia, vomiting, seizures, unconsciousness, and potentially death.

As indicated above, the presently disclosed subject-matter provide methods for the treatment of disorders associated with elevated levels of ammonia and any condition associate therewith. As used herein, “disease”, “disorder”, “condition” and the like, as they relate to a subject's health, are used interchangeably and have meanings ascribed to each and all of such terms.

It is understood that the interchangeably used terms “associated” and “related”, when referring to pathologies herein, mean diseases, disorders, conditions, or any pathologies which at least one of: share causalities, co-exist at a higher than coincidental frequency, or where at least one disease, disorder, condition or pathology causes a second disease, disorder, condition or pathology.

As noted above, the presently disclosed subject-matter provides methods for treating disorders as specified above. The term “treatment” as used herein refers to the administering of a therapeutic amount of the composition of the presently disclosed subject-matter which is effective to ameliorate undesired symptoms associated with a disease, to prevent the manifestation of such symptoms before they occur, to slow down the progression of the disease, slow down the deterioration of symptoms, to enhance the onset of remission period, slow down the irreversible damage caused in the progressive chronic stage of the disease, to delay the onset of said progressive stage, to lessen the severity or cure the disease, to improve survival rate or more rapid recovery, or to prevent the disease from occurring or a combination of two or more of the above. The treatment may be undertaken when a hemostatic condition initially develops, or may be a continuous administration, for example by administration more than once per day, every 1 day to 7 days, every 7 day to 15 days, every 15 day to 30 days, every month to two months, every two months to 6 months, or even more, to achieve the above-listed therapeutic effects.

The term “prophylaxis” refers to prevention or reduction the risk of occurrence of the biological or medical event, specifically, the occurrence or re occurrence of disorders associated with disorders associated with elevated levels of ammonia, that is sought to be prevented in a tissue, a system, an animal or a human being, by a researcher, veterinarian, medical doctor or other clinician, and the term “prophylactically effective amount” is intended to mean that amount of a pharmaceutical composition that will achieve this goal. Thus, in particular embodiments, the methods of the presently disclosed subject-matter are particularly effective in the prophylaxis, i.e., prevention of conditions associated with disorders associated with elevated levels of ammonia disorders. Thus, subjects administered with said compositions are less likely to experience symptoms associated with said disorders associated with elevated levels of ammonia disorders that are also less likely to re-occur in a subject who has already experienced them in the past.

The term “amelioration” as referred to herein, relates to a decrease in the symptoms, and improvement in a subject's condition brought about by the compositions and methods according to the presently disclosed subject-matter, wherein said improvement may be manifested in the forms of inhibition of pathologic processes associated with the disorders associated with elevated levels of ammonia disorders described herein, a significant reduction in their magnitude, or an improvement in a diseased subject physiological state.

The term “inhibit” and all variations of this term is intended to encompass the restriction or prohibition of the progress and exacerbation of pathologic symptoms or a pathologic process progress, said pathologic process symptoms or process are associated with.

The term “eliminate” relates to the substantial eradication or removal of the pathologic symptoms and possibly pathologic etiology, optionally, according to the methods of the presently disclosed subject-matter described below.

The terms “delay”, “delaying the onset”, “retard” and all variations thereof are intended to encompass the slowing of the progress and/or exacerbation of disorders associated with elevated levels of ammonia and their symptoms slowing their progress, further exacerbation or development, so as to appear later than in the absence of the treatment according to the presently disclosed subject-matter.

As noted above, treatment or prevention include the prevention or postponement of development of the disease, prevention or postponement of development of symptoms and/or a reduction in the severity of such symptoms that will or are expected to develop. These further include ameliorating existing symptoms, preventing-additional symptoms and ameliorating or preventing the underlying metabolic causes of symptoms. It should be appreciated that the terms “inhibition”, “moderation”, “reduction” or “attenuation” as referred to herein, relate to the retardation, restraining or reduction of a process, specifically, a disorders associated with elevated levels of ammonia disorder by any one of about 1% to 99.9%, specifically, about 1% to about 5%, about 5% to 10%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 60% to 65%, about 65% to 70%, about 75% to 80%, about 80% to 85% about 85% to 90%, about 90% to 95%, about 95% to 99%, or about 99% to 99.9%.

Single or multiple administrations on a daily, weekly or monthly schedule can be carried out with dose levels and pattern being selected by the treating physician. More specific embodiments relate to the use of typically 2-3 doses per week.

The presently disclosed subject-matter relates to the treatment of subjects, or patients, in need thereof. By “patient” or “subject in need” it is meant any organism who may be infected by the above-mentioned pathogens, and to whom the preventive and prophylactic products, kit/s and methods herein described is desired, including humans, domestic and non-domestic mammals such as canine and feline subjects, bovine, simian, equine and murine subjects, rodents, domestic birds, aquaculture, fish and exotic aquarium fish. It should be appreciated that the treated subject may be also any reptile or zoo animal By “mammalian subject” is meant any mammal for which the proposed therapy is desired, including human, equine, canine, and feline subjects, most specifically humans. It should be noted that specifically in cases of non-human subjects, the method of the presently disclosed subject-matter may be performed using administration via injection (intra venous (IV), intra-arterial (IA), intramuscular (IM) or subcutaneous (SC)), drinking water, feed, spraying, oral lavage and directly into the digestive tract of subjects in need thereof.

The methods discussed herein refer to the use of an effective amount. It should be understood that the terms “effective amount” or “sufficient amount” used by the methods of the invention, mean an amount necessary to achieve a selected result. More specifically, the amount of the specific conjugates as disclosed herein that is sufficient to deplete and/or remove, and/or reduce, the level of at least one amine from body fluids, specifically, to deplete or at least reduce ammonia from body fluids. Moreover, such effective amount is sufficient to provide a body fluid that comprise normal and/or non-toxic levels of ammonia. The “effective treatment amount”, as used herein, is determined by the severity of the disease in conjunction with the preventive or therapeutic objectives, the route of administration and the patient's general condition (age, sex, weight and other considerations known to the attending physician). In the context of the present invention, it refers to the effective amount of the conjugates of the present invention, used by the devices, systems, apparatus and methods disclosed herein, required for depleting and/or removing and/or reducing the levels of ammonia in body fluids, to treat, prevent, avoid and ameliorate any condition associated with elevated ammonia levels, as discussed above.

All scientific and technical terms used herein have meanings commonly used in the art unless otherwise specified. The definitions provided herein are to facilitate understanding of certain terms used frequently herein and are not meant to limit the scope of the present disclosure.

All definitions, as defined and used herein, should be understood to control over dictionary definitions, definitions in documents incorporated by reference, and/or ordinary meanings of the defined terms.

The term “about” as used herein indicates values that may deviate up to 1%, more specifically 5%, more specifically 10%, more specifically 15%, and in some cases up to 20% higher or lower than the value referred to, the deviation range including integer values, and, if applicable, non-integer values as well, constituting a continuous range. As used herein the term “about” refers to ±10%.

The indefinite articles “a” and “an,” as used herein in the specification and in the claims, unless clearly indicated to the contrary, should be understood to mean “at least one.” It must be noted that, as used in this specification and the appended claims, the singular forms “a”, “an” and “the” include plural referents unless the content clearly dictates otherwise.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc. It should also be understood that, unless clearly indicated to the contrary, in any methods claimed herein that include more than one step or act, the order of the steps or acts of the method is not necessarily limited to the order in which the steps or acts of the method are recited.

Throughout this specification and the Examples and claims which follow, unless the context requires otherwise, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures. More specifically, the terms “comprises”, “comprising”, “includes”, “including”, “having” and their conjugates mean “including but not limited to”. The term “consisting of” means “including and limited to”. The term “consisting essentially of” means that the composition, method or structure may include additional ingredients, steps and/or parts, but only if the additional ingredients, steps and/or parts do not materially alter the basic and novel characteristics of the claimed composition, method or structure.

It should be noted that various embodiments of this presently disclosed subject-matter may be presented in a range format. It should be understood that the description in range format is merely for convenience and brevity and should not be construed as an inflexible limitation on the scope of the presently disclosed subject-matter. Accordingly, the description of a range should be considered to have specifically disclosed all the possible sub ranges as well as individual numerical values within that range. For example, description of a range such as from 1 to 6 should be considered to have specifically disclosed sub ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between. As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the presently disclosed subject-matter, which are, for clarity, described in the context of separate embodiments, may also be provided in combination in a single embodiment. Conversely, various features of the presently disclosed subject-matter, which are, for brevity, described in the context of a single embodiment, may also be provided separately or in any suitable sub combination or as suitable in any other described embodiment of the presently disclosed subject-matter. Certain features described in the context of various embodiments are not to be considered essential features of those embodiments, unless the embodiment is inoperative without those elements.

Various embodiments and aspects of the presently disclosed subject-matter as delineated herein above and as claimed in the claims section below find experimental support in the following examples.

Disclosed and described, it is to be understood that this presently disclosed subject-matter is not limited to the particular examples, methods steps, and compositions disclosed herein as such methods steps and compositions may vary somewhat. It is also to be understood that the terminology used herein is used for the purpose of describing particular embodiments only and not intended to be limiting since the scope of the presently disclosed subject-matter will be limited only by the appended claims and equivalents thereof.

The following examples are representative of techniques employed by the inventors in carrying out aspects of the presently disclosed subject-matter. It should be appreciated that while these techniques are exemplary of preferred embodiments for the practice of the presently disclosed subject-matter, those of skill in the art, in light of the present disclosure, will recognize that numerous modifications can be made without departing from the spirit and intended scope of the presently disclosed subject-matter.

### EXAMPLES

Experimental Procedures

Preparation of Linker for Coupling to 4% Agarose Beads

Resin Active group: —COOH groups
Group to be coupled: —NH2 groups
45-165 μm particle size range, Ave-90 μm
Spherical, cross-linked agarose
Coupling conditions: at 4° C.-25° C., pH: 4.5-6 for 1.5-24 hrs, Coupling could be done in organic solvents.
No blocking reaction is required after the coupling reaction
Storage: 2-8° C.

Protocol:
Dissolve the linker to be coupled in the Coupling Solution, pH: 4.5-6
Preparation of resin for coupling of ligands
Wash the appropriate amount of resin five times with distilled water (pH4.5-6)
1. Add linker solution to the agarose beads in ratio 1:0.5 to 1:1 and mix gently to make suspension.
2. Add the carbodiimide solution to the suspension to a final concentration of 0.1 M.
3. Rotate the suspension end-over-end at 4° C.-RT for 4 hours
4. Adjust pH of the reaction mixture with 0.1M NaOH during first hour of the reaction.
5. Wash the resin with 0.1M acetate buffer, pH 4 containing 0.5M NaCl followed by wash with 0.1 M Tris-HCl buffer pH8 containing 0.5 M NaCl.
6. Repeat step 5 twice
7. Wash the resin with 5-10 resin volumes of distilled water if no organic solvent was used in coupling
8. following this adding sulfenic acid will done according to steps 1-7

Measuring Ammonia in Plasma

For Ammonia depletion from plasma, the plasma should be incubated with the resin in a 1:10 ratio for one hour with shaking, the ammonia particles during the incubation collide and are specifically captured by the resin. The efficacy of the incubation process examined by detecting ammonia concentration in control and incubated plasma and the depletion percentage: 100−(Incubated plasma concentration/control plasma concentration).

Ammonia concentration can be detected using Ammonia Assay Kit (Catalog #: KA0810, Manufacturer: Abnova) Ammonia or ammonium is converted to a product that reacts with the OxiRed probe to generate color (OD 570 nm) which can be easily quantified by plate reader. The kit can detect 1 nmol (˜20 μM) of ammonia or ammonium.

Established of Acute Liver Failure Model in Pigs

A midline incision was made from the xiphoid to the pubis. The portal vein was dissected free of surrounding tissue and lymph nodes from the porta hepatis to the confluence of the splenic vein. The inferior vena cava immediately cranial to the renal veins was cleared and clamped with an exclusion clamp, and a 1.5-cm longitudinal incision was made. The portal vein was then clamped, transposed, and anastomosed end-to-side to the inferior vena cava, using a continuous over-and-over polypropylene 5-0 suture. The total period of portal vein occlusion was 11-15 min. During this time and for a further 10 min, 1000 ml of 0.9% NaCl was infused into every animal to maintain the arterial pressure. The dissection of the structures in the hepatoduodenal ligament was performed carefully to ensure that also the arterial blood supply to the liver was completely interrupted. At the end of the experiment the animals are euthanized.

### Example 1

Conjugate Synthesis and Device Assembly

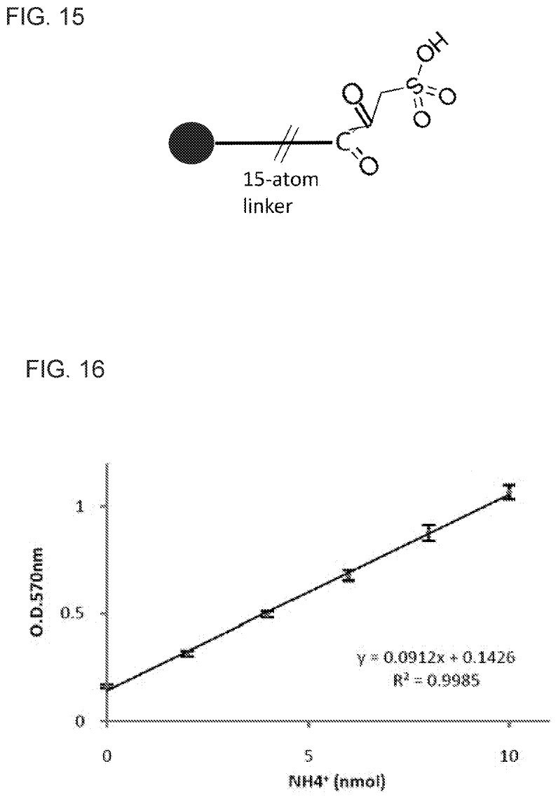

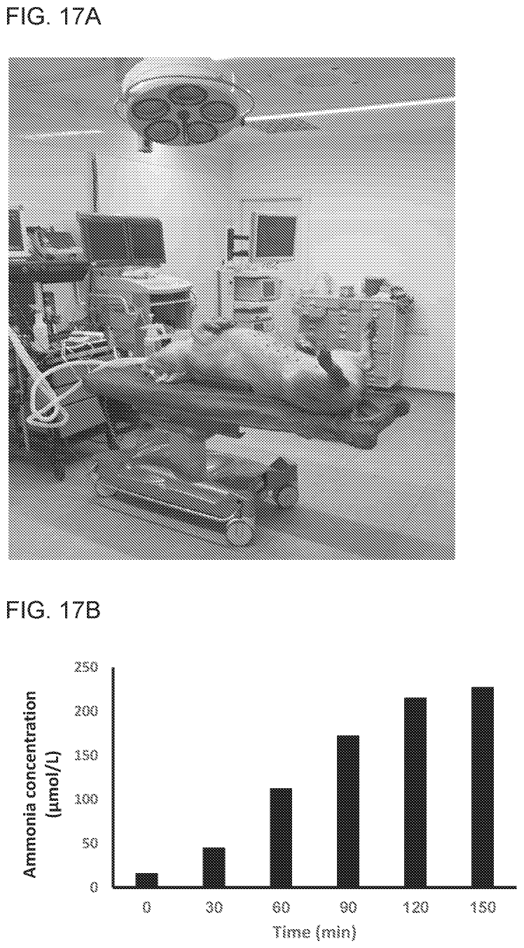

A conjugate comprising an agarose bead, a linker and sulfuric acid was prepared as indicated in the experimental procedures. More specifically, 6% agarose beads were connected to a linker of 15 carbon atoms and sulfonic acid. FIG. 15 shows a schematic presentation of the conjugate. The amount of ammonia in body fluids were calculated based on the standard curve shown in FIG. 17.

FIG. 1 represents a non-limiting example for a filter device comprising the conjugate of the invention, for use in connection with an apheresis mechanism to absorb ammonia from the blood system. The filter include resin with chemical linker, housing (plastic) and universal connection tubes that connect the filter to the apheresis mechanism.

### Example 2

Depleting Ammonium from Swine Body Fluid

Pre-clinical studies are performed in a first stage. Animal studies were performed using pigs (swine). The pigs were kept in the animal room for at least 2 days before the experiments. The conditions in the animal room are controlled (21° C., relative humidity of 30-40% and a 12:12-h light-dark cycle). The animals are fed regularly. After two days all animals were fast overnight with free access to water. The animals were looked after by the veterinarian care service, and the general health conditions are monitored continuously before the experiments. Acute liver failure (ALF) that cause increase in the ammonia levels is induced with an end-to-side portacaval shunt, followed by ligation of the hepatic arteries, and all animals are administered with saline, glucose, and albumin IV, as described by the experimental procedures. Once the ALF is initiated, the ammonia levels are monitored. FIG. 17A shows an example of the pig used in this study. The histogram in FIG. 17B shows the elevation of ammonia levels over time in the hyper Ammonia model. As shown by the figure, within 150 minutes the ammonia levels were dramatically increased up to 227 micromole per liter.

In parallel experiments, following ALF induction as described above, animals were connected to a plasma apheresis system with the device of the present disclosure, referred to herein as AAPC-300 (AMMONIA ADSORPTION PLASMA COLUMN (AAPC-300) the ammonia absorber of PlasFree), comprising the conjugate of the present disclosure to filter the ammonia during plasmapheresis. At the end of each cycle of blood filtration, the level of the ammonia is monitored and recorded. Preliminary results showed a significant reduction of the ammonia levels from 227 micromole/liter to 65 micromole/liter, thereby establishing the feasibility of the disclosed methods in for in vivo/ex vivo depletion of ammonia of a subject.

### Example 3

Depleting Ammonium from Human Blood Plasma

The inventors next evaluated the ability of the conjugate and device of the present disclosure to deplete ammonium from a unit of ammonium enriched human plasma. As shown by the illustrative scheme of FIG. 18, a human plasma bag has been connected via a flow regulator to the device of the present disclosure that contains about 270-300 ml of the conjugate disclosed herein (also designated AAPC-300). The filters plasma was collected in a bag as shown by the figure. Measurements of the ammonia levels in the filtered bag by ELISA, as presented by FIG. 19, showed a significant decrease in the ammonia levels, from an amount of about 120 micromole per litter, to about 20 micromole per litter (reduction of 6 folds).

These results therefore establish the feasibility of using the disclosed methods and devices for preparing off the shelf ammonia-free, or ammonia-reduced blood products.

1-86. (canceled)

87. A device comprising:
a housing having at least one fluid inlet port, and at least one fluid outlet port;
the housing including at least one chamber, said at least one chamber defining a control volume in fluid communication with the at least one fluid inlet port and the at least one fluid outlet port;
said control volume accommodating at least one of a conjugate, a plurality of conjugates or at least one composition comprising said conjugates or plurality of conjugates, said conjugate having the structure of formula I, the conjugate comprising a particle bonded to at least one linker comprising a chain of n carbon atoms covalently bonded to m carbonyl groups, and at least one trapping agent A covalently bonded to the m^{th} carbonyl group; 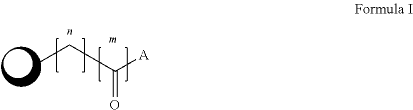
wherein, n is an integer within the range of 5 to 15, and m is an integer within the range of 5 to 10, wherein trapping agent A is characterized by having the ability to capture or bind amine;
optionally, said amine is at least one of methylamine, dimethylamine or trimethylamine.

88. The device according to claim 87, wherein at least one of:
(a) the linker comprising a straight chain alkane and m carbonyl groups; optionally,
(i) wherein the straight chain alkane is saturated or unsaturated; and/or
(ii) wherein the straight chain alkane is unsaturated; and/or
(iii) wherein the straight chain contains between 1 to 3 double bonds;
(b) the amine is ammonia;
(c) the linker is covalently linked to the trapping agent in a way that the m^{th} carbonyl is linked via straight linkage 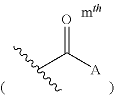
or through another short alkane chain 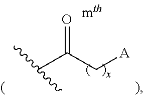
wherein X is an integer within the range of 1 to 3;
(d) the trapping agent is a strong acid, capable of capturing ammonia; optionally,
(i) wherein the strong acid is sulfuric acid or any derivates thereof; and/or
(ii) wherein the length of the straight chain alkanes (n) is 15.
(e) the device comprising a particle bonded to at least one linker comprising a chain of 15 carbon atoms covalently bonded to between 5 to 10 carbonyl groups (m), and an acid A covalently bonded to the m^{th} carbonyl group, the conjugate having the structural formula II: 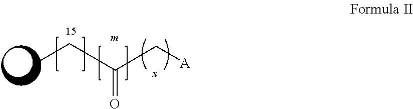
wherein x is between 0 to 3;
(f) the device comprising a particle bonded to at least one linker comprising a chain of 15 carbon atoms covalently bonded to between 5 to 10 carbonyl groups (m), and a sulfonic acid covalently bonded to the m^{th} carbonyl group, the conjugate having the structural formula III: 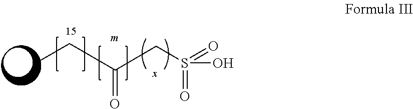
wherein x is between 0 to 3.
(g) the particle and the linker are covalently connected, and wherein the linkage is a covalent linkage via amino group as presented in Formula IV:

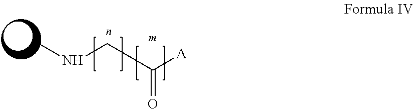

89. The device according to claim 87, having the structural formula V, the conjugate comprising a particle covalently bonded to at least one linker comprising a chain of 15 carbon atoms covalently bonded to m carbonyl groups, and a sulfonic acid covalently bonded to the m^{th} carbonyl group 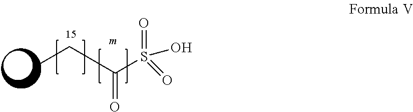
wherein m is an integer between 5 to 10.

90. The device according to claim 87, wherein at least one of:
(a) the particle is a resin bead, said resin bead optionally comprises at least 4% of agarose;
(b) the resin bead size ranges between 40 to 170 μm;
(c) the device comprises a first barrier member and a second barrier member, longitudinally spaced from one another via said control volume, the first barrier member and the second barrier member each being configured for permitting fluid flow in one direction through the respective barrier member, and for blocking fluid flow in an opposite direction through the respective barrier member; optionally, at least one of:
(i) the first barrier member and the second barrier member are installed in the device in a manner to permit fluid flow through the device from the at least one fluid inlet port to the at least one fluid outlet port, and for concurrently blocking fluid flow from the fluid outlet port to the fluid inlet port; and/or
(ii) each one of the first barrier member and the second barrier member comprises a membrane made from suitable material.

91. The device according to claim 87, wherein the housing comprises an outer casing, an inlet end cap and an outlet end cap, wherein the outer casing comprises an outer wall extending longitudinally between an inlet end and an outlet end of the outer casing, wherein the inlet end cap is configured for being sealingly mounted to the inlet end, and wherein the outlet end cap is configured for being sealingly mounted to the outlet end; optionally, at least one of:
(a) wherein inlet end cap, the outlet end cap, and the outer casing are each made from suitable medically compatible materials;
(b) wherein the inlet end cap is configured as a self-locking cap with respect to the outer casing, and configured for enabling the inlet end cap to be sealingly locked in place with respect to the outer casing; optionally, the device comprising a first self-locking arrangement configured for enabling self-locking of the inlet end cap with respect to the outer casing.

92. The device according to claim 91, wherein the first self-locking arrangement comprises a plurality of first wedge elements and a first flange arrangement, wherein the first wedge elements are provided in the inlet end cap, and wherein the first flange arrangement is provided in the outer casing longitudinally spaced from the inlet end by a first spacing, and wherein the first wedge elements are configured for cooperating with a first flange stop arrangement to provide self-locking of the inlet end cap with respect to the housing; optionally, wherein at least one of:
(a) each said first wedge element is projecting in a longitudinal direction away from a free end of the first end cap;
(b) the first spacing is sufficient such as to ensure that when the inlet end cap is fully engaged with the outer casing, the respective free end of the inlet end cap is in abutting contact with the first flange arrangement; and
(c) the first flange stop arrangement comprises a plurality of first stop elements corresponding to the plurality of first wedge elements, and wherein each said first stop element operates to prevent disengagement of the inlet end cap from the outer casing when the respective first wedge element is in abutting contact therewith, optionally, the first flange stop arrangement comprises a first flange including a plurality of first cutouts corresponding to the first wedge elements, and wherein each said first cutout has a circumferential length and an axial depth sufficient to enable accommodating a respective said first wedge element therein in locked configuration.

93. The device according to claim 91, wherein the outlet end cap is configured as a self-locking cap with respect to the outer casing, and configured for enabling the outlet end cap to be sealingly locked in place with respect to the outer casing; optionally, at least one of:
(a) the device comprising a second self-locking arrangement configured for enabling self-locking of the outlet end cap with respect to the outer casing;
(b) the second self-locking arrangement comprises a plurality of second wedge elements and a second flange arrangement, wherein the second wedge elements are provided in the outlet end cap, and wherein the second flange arrangement is provided in the outer casing longitudinally spaced from the outlet end by a second spacing, and wherein the second wedge elements are configured for cooperating with a second flange stop arrangement to provide self-locking of the outlet end cap with respect to the housing; optionally, at least one of:
(i) each said second wedge element is projecting in a longitudinal direction away from a free end of the second end cap; and/or
(ii) wherein the second spacing is sufficient such as to ensure that when the outlet end cap is fully engaged with the outer casing, the respective free end of the outlet end cap is in abutting contact with the second flange arrangement; and/or
(iii) wherein the second flange stop arrangement comprises a plurality of second stop elements corresponding to the plurality of second wedge elements, and wherein each said second stop element operates to prevent disengagement of the outlet end cap from the outer casing when the respective second wedge element is in abutting contact therewith; and/or
(iv) wherein the second flange stop arrangement comprises a second flange including a plurality of second cutouts corresponding to the second wedge elements, and wherein each said second cutout has a circumferential length and an axial depth sufficient to enable accommodating a respective said second wedge element therein in locked configuration.

94. The device according to claim 87, wherein at least one of:
(a) said control volume is between about 250 ml and about 350 ml;
(b) the device is for use in depleting at least one amine from at least one liquid substance; optionally, at least one of:
(i) wherein said amine is ammonia; and/or
(iii) wherein said liquid substance is a mammalian body fluid, said device is for use in depleting ammonia from mammalian body fluid/s; and
(c) wherein said conjugate is having the structural formula V, the conjugate comprising a particle covalently bonded to at least one linker comprising a chain of 15 carbon atoms covalently bonded to m carbonyl groups, and a sulfonic acid covalently bonded to the m^{th} carbonyl group 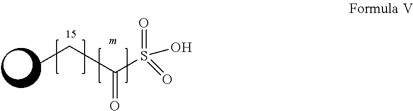
wherein m is an integer between 5 to 10.

95. A system comprising:
at least one device as defined in claim 87;
an apheresis machine;
a blood mixing reservoir; and
a conduit system.

96. The system according to claim 95, wherein at least one of:
(a) said conduit system comprises a first conduit configured for providing selective fluid communication between the apheresis machine and a body of a subject in need thereof, to thereby enable blood to flow to the apheresis machine from the body of a subject in need thereof;
(b) said conduit system comprises a second conduit configured for providing fluid communication from a plasma outlet of the apheresis machine and the at least one device, to thereby enable plasma, separated from blood by the apheresis machine, to flow into the at least one device;
(c) said conduit system comprises a third conduit configured for providing fluid communication from the at least one device to the blood mixing reservoir, to thereby enable processed plasma, treated by the at least one device, to flow into the blood mixing reservoir;
(d) said conduit system comprises a fourth conduit configured for providing fluid communication from a blood products outlet of the apheresis machine to the blood mixing reservoir, to thereby enable other blood products separated from blood by the apheresis machine, to flow into the blood mixing reservoir;
(e) said conduit system comprises a fifth conduit configured for providing selective fluid communication between the blood mixing reservoir and the body of a subject in need thereof, to thereby enable treated blood to flow from the blood mixing reservoir to the body of a subject in need thereof;
(f) the system comprising a plurality of said devices, interconnected in series with respect to one another;
(g) the system comprising a plurality of said devices, interconnected in parallel with respect to one another via an inlet manifold coupled to each respective fluid inlet port, and via an outlet manifold coupled to each respective fluid outlet port; and
(h) the system comprising a first plurality of groups of said devices, the groups being interconnected in parallel with respect to one another via an inlet manifold coupled to each respective fluid inlet port, and via an outlet manifold coupled to each respective fluid outlet port, and wherein each said group comprising a respective second plurality of said devices interconnected in series with respect to one another within the respective group.

97. A battery for use in depleting ammonia from mammalian body fluid/s, comprising a plurality of devices as defined by claim 87.

98. An extracorporeal apparatus comprising at least one conjugate, or at least one device comprising said conjugate, or connected to said at least one device or battery of devices, wherein said conjugate comprising a particle bonded to at least one linker comprising a chain of n carbon atoms covalently bonded to m carbonyl groups, and at least one trapping agent A covalently bonded to the m^{th} carbonyl group, 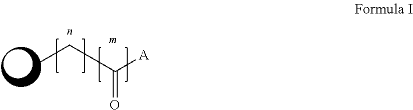
wherein, n is an integer within the range of 5 to 15, and m is an integer within the range of 5 to 10, wherein trapping agent A is characterized by having the ability to capture or bind amine;
optionally, said amine is at least one of methylamine, dimethylamine or trimethylamine, and wherein said device comprising:
a housing having at least one fluid inlet port, and at least one fluid outlet port;
the housing including at least one chamber, said at least one chamber defining a control volume in fluid communication with the at least one fluid inlet port and the at least one fluid outlet port;
said control volume accommodating said at least one of a conjugate, a plurality of conjugates or at least one composition comprising said conjugates or plurality of conjugates, optionally, said extracorporeal apparatus is for use in depleting ammonia from mammalian body fluid/s.

99. A conjugate having the structural formula I, the conjugate comprising a particle bonded to at least one linker comprising a chain of n carbon atoms covalently bonded to m carbonyl groups, and at least one trapping agent A covalently bonded to the m^{th} carbonyl group; 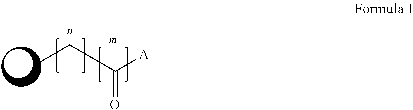
wherein, n is an integer within the range of 5 to 15, and m is an integer within the range of 5 to 10, wherein trapping agent A is characterized by having the ability to capture or bind amine, optionally, said amine is at least one of methylamine, dimethylamine or trimethylamine.

100. The conjugate according to claim 99, wherein at least one of:
(a) the linker comprising a straight chain alkane and m carbonyl groups; optionally, at least one of:
(i) wherein the straight chain alkane is saturated or unsaturated; and/or
(ii) wherein the straight chain alkane is unsaturated; and/or
(iii) wherein the straight chain contains between 1 to 3 double bonds;
(b) wherein the amine is ammonia;
(c) wherein the linker is covalently linked to the trapping agent in a way that the m^{th} carbonyl is linked via straight linkage 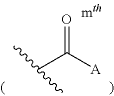
or through another short alkane chain 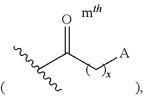
wherein X is an integer within the range of 1 to 3;
(d) wherein the trapping agent is a strong acid, capable of capturing ammonia; optionally, wherein at least one of:
(i) wherein the strong acid is sulfuric acid or any derivates thereof; and/or
(ii) wherein the length of the straight chain alkanes (n) is 15.

101. The conjugate according to claim 99, wherein at least one of:
(a) the conjugate comprising a particle bonded to at least one linker comprising a chain of 15 carbon atoms covalently bonded to between 5 to 10 carbonyl groups (m), and an acid A covalently bonded to the m^{th} carbonyl group, the conjugate having the structural formula II: 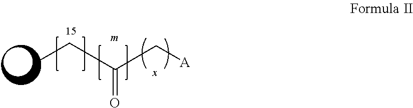
wherein x is between 0 to 3;
(b) the conjugate comprising a particle bonded to at least one linker comprising a chain of 15 carbon atoms covalently bonded to between 5 to 10 carbonyl groups (m), and a sulfonic acid covalently bonded to the m^{th} carbonyl group, the conjugate having the structural formula III: 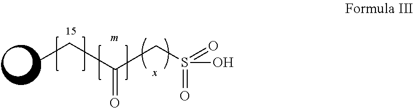
wherein x is between 0 to 3; and
(c) wherein the particle and the linker and covalently connected, and wherein the linkage is a covalent linkage via amino group as presented in Formula IV:

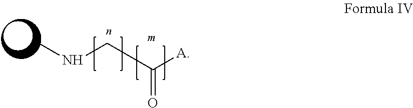

102. The conjugate according to claim 99, having the structural formula V, the conjugate comprising a particle covalently bonded to at least one linker comprising a chain of 15 carbon atoms covalently bonded to m carbonyl groups, and a sulfonic acid covalently bonded to the m^{th} carbonyl group 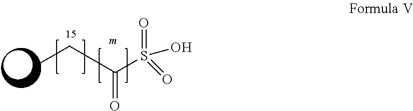
wherein m is an integer between 5 to 10; optionally, at least one of:
(a) wherein the particle is a resin bead, said resin bead optionally comprises at least 4% of agarose; and
(b) wherein the resin bead size ranges between 40 to 170 μm.

103. A plurality of conjugates as defined in claim 99, or any composition comprising said plurality of conjugates, wherein each conjugate comprises a particle, at least one linker and at least one trapping agent A, or any derivative or analog thereof, the conjugate comprising a particle bonded to at least one linker comprising a chain of n carbon atoms covalently bonded to m carbonyl groups, and at least one trapping agent A covalently bonded to the m^{th} carbonyl group; 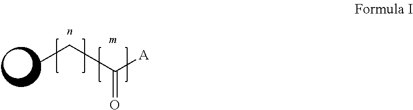
wherein, n is an integer within the range of 5 to 15, and m is an integer within the range of 5 to 10, wherein trapping agent A is characterized by having the ability to capture or bind amine, optionally, said amine is at least one of methylamine, dimethylamine or trimethylamine;
optionally, at least one of:
the plurality of conjugates is for use in depleting at least one amine from at least one liquid substance; and optionally at least one of:
(i) wherein said amine is ammonia; and
(ii) wherein said liquid substance is a mammalian body fluid, said plurality of conjugates is for use in depleting ammonia from mammalian body fluid/s.

104. A method for depleting at least one amine from a liquid substance, the method comprising the steps of:
(i) subjecting said liquid substance to affinity-depletion procedure specific for said at least one amine; and
(ii) recovering the at least one amine-depleted liquid obtained in step (i);
wherein said affinity-depletion procedure comprises contacting said liquid substance with an effective amount of at least one conjugate as defined in claim 99, a plurality of conjugates or with a composition comprising said conjugate or plurality of conjugates, or applying said liquid substance on a device, battery, or extracorporeal apparatus comprising said conjugates, wherein each conjugate comprises a particle bonded to at least one linker comprising a chain of n carbon atoms covalently bonded to m carbonyl groups, and at least one trapping agent A covalently bonded to the m^{th} carbonyl group, 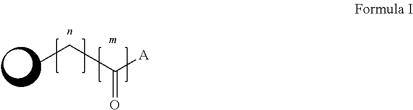
wherein, n is an integer within the range of 5 to 15, and m is an integer within the range of 5 to 10, wherein trapping agent A is characterized by having the ability to capture or bind said amine;
optionally, said amine is at least one of methylamine, dimethylamine or trimethylamine;
optionally, at least one of:
(a) wherein said liquid substance is a mammalian body fluid/s or any products thereof;
and/or
(b) wherein said at least one amine is ammonia, said method is for use in depleting ammonia from a mammalian body fluid.

105. A method for depleting at least one amine from body fluid/s of a subject in need thereof by an extracorporeal procedure, the method comprising the steps of:
(i) transferring body fluids of said subject into an extracorporeal apparatus;
(ii) subjecting said body fluid to affinity depletion procedure specific for at least one amine, wherein said depletion is performed before, during or after blood is being transferred into and out-off said apparatus, thereby obtaining an extracorporeal body fluid of said subject depleted in at least one amine; and
(iii) reintroducing or returning said body fluid obtained in step (ii) to said subject;
wherein said affinity-depletion procedure comprises contacting said body fluid with an effective amount of conjugates, a plurality of conjugates or a composition thereof comprised within said extracorporeal apparatus, or within a device or battery connected to said extracorporeal apparatus, wherein each conjugate comprises a particle bonded to at least one linker comprising a chain of n carbon atoms covalently bonded to m carbonyl groups, and at least one trapping agent A covalently bonded to the m^{th} carbonyl group, 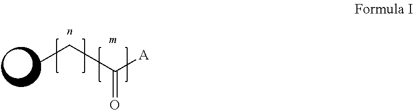
wherein, n is an integer within the range of 5 to 15, and m is an integer within the range of 5 to 10, wherein trapping agent A is characterized by having the ability to capture or bind amine;
optionally, said amine is at least one of methylamine, dimethylamine or trimethylamine, optionally, wherein said conjugate is having the structural formula V, the conjugate comprising a particle covalently bonded to at least one linker comprising a chain of 15 carbon atoms covalently bonded to m carbonyl groups, and a sulfonic acid covalently bonded to the m^{th} carbonyl group 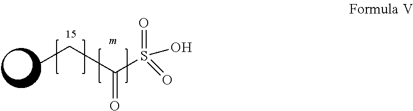
wherein m is an integer between 5 to 10.

106. A method for the treatment, prevention, prophylaxis, amelioration, inhibition of disorders associated with elevated blood ammonia levels or pathologic condition associated therewith in a subject in need thereof by depleting ammonia from body fluid/s of a subject in need thereof by an extracorporeal procedure, the method comprising the steps of:
a. transferring body fluids of said subject into an extracorporeal apparatus;
b. subjecting said body fluid to affinity depletion procedure specific for said ammonia, wherein said depletion is performed before, during or after blood is being transferred into and out-off said apparatus, thereby obtaining an extracorporeal body fluid of said subject depleted in ammonia; and
c. reintroducing or returning said body fluid obtained in step (b) to said subject;
wherein said affinity-depletion procedure comprises contacting said body fluid with an effective amount of conjugate(s) as defined in claim 99, a plurality of conjugates or a composition thereof comprised within said extracorporeal apparatus, or within a device or battery connected to said extracorporeal apparatus, wherein each conjugate comprises a particle bonded to at least one linker comprising a chain of n carbon atoms covalently bonded to m carbonyl groups, and at least one trapping agent A covalently bonded to the m^{th} carbonyl group, 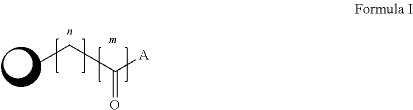
wherein, n is an integer within the range of 5 to 15, and m is an integer within the range of 5 to 10, wherein trapping agent A is characterized by having the ability to capture or bind ammonia;
optionally, wherein at least one of:
(i) said conjugate is having the structural formula V, the conjugate comprising a particle covalently bonded to at least one linker comprising a chain of 15 carbon atoms covalently bonded to m carbonyl groups, and a sulfonic acid covalently bonded to the m^{th} carbonyl group 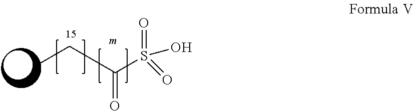
wherein m is an integer between 5 to 10; and
(ii) said disorders associated with elevated blood ammonia levels is a chronic hepatic or pulmonary condition and/or cognitive decline, and/or hyperammonemia and associated conditions, optionally, said hepatic condition is hepatic encephalopathy and any related conditions.
